(19) United States
(12) Patent Application Publication
(10) Pub. No.: US 2024/0226809 A9
(43) Pub. Date: Jul. 11, 2024

(54) METHODS OF ISOLATING EXOSOMES

(71) Applicant: VIRGINIA POLYTECHNIC INSTITUTE AND STATE UNIVERSITY, BLACKSBURG, VA (US)

(72) Inventors: Spencer MARSH, Roanoke, VA (US); Kevin J. PRIDHAM, Roanoke, VA (US); Linda Jane JOURDAN, Roanoke, VA (US); Robert G. GOURDIE, Roanoke, VA (US)

(73) Assignee: VIRGINIA TECH INTELLECTUAL PROPERTIES, INC., BLACKSBURG, VA (US)

(21) Appl. No.: 18/547,489

(22) PCT Filed: Feb. 23, 2022
(86) PCT No.: PCT/US2022/017554

§ 371 Date: Aug. 22, 2023

Related U.S. Application Data
(60) Provisional application No. 63/152,784, filed on Feb. 23, 2021.

(51) Int. Cl. B01D 61/14

(52) U.S. Cl. CPC B01D 61/14; B01D 2311/2676; B01D 2315/10; B01D 2315/16

(57) ABSTRACT

Described in certain example embodiments herein are methods of isolating exosomes from a biological fluid, such as those containing caseins.

### CROSS-REFERENCE TO RELATED APPLICATIONS

This application claims the benefit of and priority to co-pending U.S. Provisional Patent Application No. 63/152,784, filed on Feb. 23, 2021, entitled “METHODS OF ISOLATING EXOSOMES,” the contents of which is incorporated by reference herein in its entirety.

### STATEMENT REGARDING FEDERALLY SPONSORED RESEARCH

This invention was made with government support under Grant No.(s) HL161237; HL056728; and HL141855 awarded by the National Institutes of Health. The government has certain rights in the invention.

### TECHNICAL FIELD

The subject matter disclosed herein is generally directed to isolation and preparation of exosomes.

### BACKGROUND

Exosomes are membrane-bound nanovesicles released by cells that act as an evolutionarily conserved mechanism for long-range intercellular signaling (Boulanger, 2017). In humans and other mammals, exosomes are secreted into the extracellular environment by nearly all cell types and are abundant in most biological fluids including blood, lymph, urine and milk (Gyorgy, B. S. (2011). Membrane vesicles, current state-of-the-art: emerging role of extracellular vesicles. Cellular and molecular life sciences, 2667). Exosomes are of relatively uniform small size, being 50-150 nm in diameter, and show preferential expression of a number of proteins, including CD81, CD9 and syntenin, but not others such as calnexin (Vlassov, A. M. (2012). Exosomes: Current knowledge of their composition, biological functions, and diagnostic and therapeutic potentials. BBA-General Subjects, 940-947). This being said, exosomal constituents can vary considerably, reflecting the type and/or physiological state of the cells from which they were secreted (Blaser MC, A. E. (2018). Roles and Regulation of Extracellular Vesicles in Cardiovascular Mineral Metabolism. Front Cardiovasc Med.). Exosomal cargos include lipids, proteins, and nucleotide sequences (e.g., microRNAs), which can be internally encapsulated or present as external moieties such as receptors or adhesion molecules on the vesicular membrane (Rana, S. Z. (2011). Exosome target cell selection and the importance of exosomal tetraspanins: a hypothesis. Biochem Soc Trans, 559-562). The ability of exosomes to transport and protect biological signaling molecules in vivo has attracted the attention of the pharmaceutical industry as it has become apparent that they could be utilized as a drug delivery platform. This appeal is further enhanced by the unique ability of certain exosomal populations to cross tissue boundaries such as the cutaneous barrier (Carrasco E, S.-H. G. (2019). The role of extracellular vesicles in Cutaneous Remodeling and Hair Follicle Dynamics. Int J Mol Sci, 2758), blood-brain-barrier (Yang T, M. P. (2015). Exosome delivered anticancer drugs across the blood-brain barrier for brain cancer therapy in Danio rerio. Pharm Res, 2003-2014), and gut-blood barrier (Vashisht M, R. P. (2017). Curcumin encapsulated in milk exosomes resists human digestion and possesses enhanced intestinal permeability in vitro. Applied Biochem Biotechnol, 993-1007). Exosomes also appear to elude immune surveillance and have been reported to be immunologically well-tolerated even when transferred autologously between individuals and species (Antes T J, M. R. (2018). Targeting extracellular vesicles to injured tissue using membrane cloaking and surface display. J Nanobiotechnology)—further heightening interest in their potential for translation to the clinic as a novel means for improving the safety of drug delivery. A limitation to the clinical and practical use of exosomes is that methods of cost-effective purification, particularly in large quantities, is unavailable. As such there exists a need for improved methods of exosome isolation, particularly those capable of scaling for industrial scale exosome preparation.

Citation or identification of any document in this application is not an admission that such a document is available as prior art to the present invention.

### SUMMARY

Described in certain example embodiments herein are methods of isolating exosomes (also referred to as small extracellular vesicles) from a biological fluid, the method comprising centrifuging a biological fluid under conditions suitable to separate fats from one or more other components of the biological fluid; removing the separated fats from the biological fluid; after step (b) centrifuging the remaining biological fluid one or more times and skimming any noticeable separated fats after each centrifuging in step (c); filtering the remaining biological fluid after step (c) optionally performing one or more ultracentrifugation steps after (d); chelating divalent cations with about 10 mM to about 100 mM EDTA at about 30-42 degrees Celsius after (d) or optionally (e) and optionally for about 15-120 minutes; and after (f), optionally performing tangential flow filtration to obtain a retentate, wherein the retentate is optionally ultracentrifuged via one or more ultracentrifugation steps or stored at −80 degrees C., and optionally fractionating the retentate, optionally via column separation, after the retentate is optionally ultracentrifuged or stored at −80 degrees C., wherein the method comprises step (e) or step (g) but not both.

In certain example embodiments, chelating divalent cations occurs with about 30 mM EDTA.

In certain example embodiments, chelating divalent cations occurs at about 37 degrees Celsius.

In certain example embodiments, chelation of divalent ions at about 37 degrees occurs for 60 minutes.

In certain example embodiments, the biological fluid is mammalian milk.

In certain example embodiments, the biological fluid is unpasteurized.

In certain example embodiments, steps (a) and (b) together are repeated 1-5 times.

In certain example embodiments, step (a), (b), (c), (d), (e), (g), or any combination thereof is performed at about 4 degrees Celsius.

In certain example embodiments, (a) comprises centrifuging the biological fluid at about 2,000-3,000 rcf In certain example embodiments, (a) comprises centrifuging the biological fluid at about 2,500 rcf.

In certain example embodiments, step (a) is repeated 1-3 times.

In certain example embodiments, (b) comprises a first centrifugation followed by a second centrifugation. In certain example embodiments, the first centrifugation comprises centrifuging the remaining biological fluid at about 13,500-15,500 rcf for about 45-75 minutes. In certain example embodiments, the first centrifugation comprises centrifuging the remaining biological fluid at about 14,500 rcf for about 60 minutes. In certain example embodiments, the second centrifugation is performed on the biological fluid remaining after the first centrifugation and wherein the second centrifugation is performed at about 24,800-26,800 rcf for about 45-75 minutes. In certain example embodiments, the second centrifugation is performed on the biological fluid remaining after the first centrifugation and wherein the second centrifugation is performed at about 25,800 rcf for about 60 minutes. In certain example embodiments, the second centrifugation is repeated 1-3 times with each repetition being performed on the remaining biological fluid from the centrifugation immediately prior.

In certain example embodiments, (d) comprises filtering the remaining biological fluid through one or more filters in series ranging from about a 0.45 micron filter to about a 0.22 micron filters. In certain example embodiments, (d) comprises filtering the remaining biological fluid through an about 0.45 micron filter followed by filtering the remaining biological fluid through an about 0.22 micron filter.

In certain example embodiments, (e) comprises 2 or more serial ultracentrifugation steps, wherein each step is performed on the remaining biological fluid from the prior ultracentrifugation. In certain example embodiments, (e) comprises an ultracentrifugation step performed at about 45,000-55,000 rcf, an ultracentrifugation step performed at about 65,000-75,000 rcf, an ultracentrifugation step performed at about 90,000-110,000 rcf, or a combination thereof. In certain example embodiments, (e) comprises an ultracentrifugation step performed at about 50,000 rcf, an ultracentrifugation step performed at about 70,000 rcf, an ultracentrifugation step performed at about 100,000 rcf, or a combination thereof. In certain example embodiments, the one or more of the one or more ultracentrifugation steps are each performed for about 45-75 minutes. In certain example embodiments, the one or more of the one or more ultracentrifugation steps are each performed for about 60 minutes.

In certain example embodiments, (e) comprises a final ultracentrifugation step performed at about 115,000-145,000 rcf, for about 90-150 minutes and wherein the resulting fluid is discarded, and the remaining pellet is resuspended in a suitable volume of a suitable solution prior to (f). In certain example embodiments, (e) comprises a final ultracentrifugation step performed at about 130,000 rcf, for about 120 minutes and wherein the resulting fluid is discarded, and the remaining pellet is resuspended in a suitable volume of a suitable solution prior to (f).

In certain example embodiments, the tangential flow filtration of (g) is performed using ultrafiltration membrane with a cutoff ranging from about 250 kDa to about 750 kDa. In certain example embodiments, the tangential flow filtration of (g) is performed using a 250 kDa ultrafiltration membrane.

In certain example embodiments, the tangential flow filtration of (g) is performed at a flow rate of about 5-15 mL per minute. In certain example embodiments, the tangential flow filtration of (g) is performed at a flow rate of about 10 mL per minute.

In certain example embodiments, in step (g), when the amount of remaining biological fluid reaches about ten percent of its starting volume before tangential flow filtration the retentate is diafiltered with a suitable buffer.

In certain example embodiments, the method further comprises ultracentrifuging the retentate when the retentate reaches about 20 percent of the starting diafiltration amount. In certain example embodiments, the ultracentrifugation is performed at about 115,000-145,000 rcf for about 90-150 minutes at about 4 degrees Celsius. In certain example embodiments, the ultracentrifugation is performed at about 130,000 rcf for about 120 minutes at about 4 degrees Celsius.

In certain example embodiments, the method does not include ultracentrifuging the retentate when the retentate reaches about 20 percent of the starting diafiltration amount. In certain example embodiments, the retentate is stored at −80 degrees C. after the retentate reaches about 20 percent of the starting diafiltration amount and prior to column separation.

In certain example embodiments, the method yields an exosomal concentrate that is at least 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or at least 20 percent of the starting volume of milk.

In certain example embodiments, the method further comprises loading the exosomes of the formulation resulting from the method of any one of the preceding claims, with one or more cargos.

Described in certain example embodiments herein are formulations, optionally pharmaceutical formulations, where the formulation is produced at least in part or in whole by a method described herein. In certain example embodiments, one or more of the exosomes are loaded with one or more cargos.

Described in certain example embodiments herein are methods comprising administering a formulation described herein, such as one comprising an exosome and/or that is produced by a method of exosome isolation described herein, to a subject in need thereof. In certain example embodiments, the one or more cargos are therapeutic cargos.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Overview of steps in an embodiment of an ultracentrifugation-based method of exosome isolation from milk.

FIG. 2—Overview of steps in an embodiment of a tangential flow filtration-based method of exosome isolation from milk.

FIGS. 3A-3E—An embodiment of an ultracentrifuge based protocol for exosome purification. (FIG. 3A) Sequential nanodrop fractions collected during this SEC filtration step, with protein concentrations in mg/ml on the y axis. (FIG. 3B) Western blot of exosomal markers CD-81, CD-9 and Syntenin, along with non-exosomal markers casein, and Arf6 (microvesicular marker) and Calnexin (cell membrane marker). Peak exosome SEC fractions occur between fractions 8 and 9. Contaminating proteins, including casein, predominate after fraction 12. Lysates from HeLa cells are included as comparative controls. (FIG. 3C) Nanosight Tracker analysis data for exosome isolates. (FIG. 3D) Negative stain electron microscopy of final exosomal and (FIG. 3E) casein isolates.

FIGS. 4A-4D—Overview of analysis of TFF based protocol. (FIG. 4A) Nanodrop results from optimized TFF-UC protocol indicating associated Exosomal and Protein fractions. (FIG. 4B) Western blot analysis of exosome markers CD-81, CD-9 and Syntenin, along with interfering protein marker Casein, and Microvesicle marker Arf6 and Calnexin. These results indicate highly pure exosomes without interfering protein or microvesicles. (FIG. 4C) Nanosight Tracker analysis data for exosome isolates. Concentration shown under NTA analysis (FIG. 4D) Negative stain electron microscopy of final isolates, showing ultra-dense accumulation of exosomes in peak SEC fractions and (FIG. 4D) high levels of casein macrostructures in peak protein fraction

FIG. 5—Calcein uptake into isolated milk exosomes. Peak exosome containing SEC fractions generated by the TFF-based method diluted 1:10 in Hepes buffer. The images show uptake resulting from 1, 2, 3 and 4 hour incubations in Calcein-AM. The uptake of dye suggests that the extracellular vesicles contain esterase activity and are capable of retaining de-esterified Calcein molecules.

FIG. 6—Representative photographic and TEM mages demonstrating isolation of milk exosomes. Upper left photographic image shows a starting volume (1L) of milk and bottom left photographic image shows a typical post-isolation volume of about 125 mL of an exosomal concentrate acquired via TFF-based exosome isolation. The right TEM image shows a representative, high magnification image of TFF isolated exosomes. The inset TEM image shows a high mag of a standard exosome. The representative TEM image is post-SEC, exosome contain fraction number 8.5 of FIG. 4A.

FIGS. 7A-7B—Overview of loss of exosomes during ultracentrifugation protocol. High numbers of exosomes are lost during UC protocol, highlighting need for reduction in UC spins and the incorporation of TFF into procedure. FIG. 7A depicts histogram of SEC separated 100,000 rcf pellet, with negatively stained EM images for peak fractions 8.0-9.0 shown below. FIG. 7B depicts histogram of SEC separated 70,000 rcf pellet, with negatively stained EM images for peak fractions 8.0-9.0 shown below.

FIG. 8—Overview of loss of exosomes during ultracentrifugation process, post-130,000 RCF spin. High number of exosomes are left in the supernatant at the end of the UC protocol, highlighting the need for TFF reduction in interfering protein in order to optimize the efficiency.

FIGS. 9A-9D—Effects of different aspects of temperature assisted chelation on exosomal samples. (FIG. 9A) Raw “gold standard” conventional UC isolated exosomes-insert shows high mag of casein micellar structure. (FIG. 9B) Full “gold standard” conventional isolation coupled with EDTA treatment unassisted by either SEC or TFF filtration. (FIG. 9C) Results with the modified UC protocol of the present disclosure subjected to 1 hour at 37° C. and SEC filtration with no EDTA treatment. (FIG. 9D) Results from the modified UC protocol of the present disclosure treated with 30 mM EDTA at room temperature (20° C.) and SEC filtration.

FIGS. 10—100 mM EDTA treatment at 37° C. results in overall exosome loss as well as damage to the exosome ultrastructure as shown by black boxes, coupled with reduced efficiency in exosome isolation.

FIGS. 11A-11D—Isolation of exosomes from human breast milk via an embodiment of an ultracentrifugation isolation method. FIG. 11A shows a graph of the concentration of exosomes or protein in each fraction (x-axis) in mg/mL (y-axis). FIG. 11B shows Nanosight Tracker analysis data for exosome isolates. FIGS. 11C-11D show a high magnification TEM images of isolated exosomes (FIG. 11C) and representative TEM images of various exosome fractions (FIG. 11D).

FIG. 12—Representative TEM images demonstrating the effect of storage post-SEC on isolated exosomes. Storage conditions are specified under TEM images

FIGS. 13A-13B—Representative TEM images of isolated exosomes after storage pre SEC. FIG. 13A shows fresh isolated exosomes and no storage at —80 degrees C. FIG. 13B shows isolated exosomes stored at —80 degrees C. post-TFF and Pre-SEC for 6 months prior to SEC processing.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes I X, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Exosomes are membrane-bound nanovesicles released by cells that act as an evolutionarily conserved mechanism for long-range intercellular signaling (Boulanger, 2017). In humans and other mammals, exosomes are small extracellular vesicles that are secreted into the extracellular environment by nearly all cell types and are abundant in most biological fluids including blood, lymph, urine and milk (Gyorgy, B. S. (2011). Membrane vesicles, current state-of-the-art: emerging role of extracellular vesicles. Cellular and molecular life sciences, 2667). Exosomes are of relatively uniform small size, being 50-150 nm in diameter, and show preferential expression of a number of proteins, including CD81, CD9 and syntenin, but not others such as calnexin (Vlassov, A. M. (2012). Exosomes: Current knowledge of their composition, biological functions, and diagnostic and therapeutic potentials. BBA-General Subjects, 940-947). This being said, exosomal constituents can vary considerably, reflecting the type and/or physiological state of the cells from which they were secreted (Blaser M C, A. E. (2018). Roles and Regulation of Extracellular Vesicles in Cardiovascular Mineral Metabolism. Front Cardiovasc Med.). Exosomal cargos include lipids, proteins, and nucleotide sequences (e.g., microRNAs), which can be internally encapsulated or present as external moieties such as receptors or adhesion molecules on the vesicular membrane (Rana, S. Z. (2011). Exosome target cell selection and the importance of exosomal tetraspanins: a hypothesis. Biochem Soc Trans, 559-562). The ability of exosomes to transport and protect biological signaling molecules in vivo has attracted the attention of the pharmaceutical industry as it has become apparent that they could be utilized as a drug delivery platform. This appeal is further enhanced by the unique ability of certain exosomal populations to cross tissue boundaries such as the cutaneous barrier (Carrasco E, S.-H. G. (2019). The role of extracellular vesicles in Cutaneous Remodeling and Hair Follicle Dynamics. Int J Mol Sci, 2758), blood-brain-barrier (Yang T, M. P. (2015). Exosome delivered anticancer drugs across the blood-brain barrier for brain cancer therapy in Danio rerio. Pharm Res, 2003-2014), and gut-blood barrier (Vashisht M, R. P. (2017). Curcumin encapsulated in milk exosomes resists human digestion and possesses enhanced intestinal permeability in vitro. Applied Biochem Biotechnol, 993-1007). Exosomes also appear to elude immune surveillance and have been reported to be immunologically well-tolerated even when transferred autologously between individuals and species (Antes T J, M. R. (2018). Targeting extracellular vesicles to injured tissue using membrane cloaking and surface display. J Nanobiotechnology)—further heightening interest in their potential for translation to the clinic as a novel means for improving the safety of drug delivery.

There are a number of methods for exosomal isolation from biological fluids, with the current “gold standard” techniques being based on ultracentrifugation (UC). These methods typically involve differential centrifugation steps and/or density gradient UC-based separations. However, the ability to produce exosomes in large quantities is restricted by the requirement for multiple UC steps and the fact that UCs can only spin small volumes. It further remains that shearing forces imparted during repeated UC spins may have deleterious effects on exosome structural integrity (Taylor D D, S. S. (2015). Methods of isolating extracellular vesicles impact down-stream analyses of their cargoes. Methods, 3-10). Other techniques that may exert less physical rigor during exosomal isolation include ultrafiltration, tangential flow filtration (TFF), size exclusion chromatography (SEC), and polyethylene glycol precipitation based methods. The use of each has been well documented, with the majority of groups utilizing a mixture of these approaches as opposed to one standalone method (Pin Li, M. K. (2017). Progress in exosome isolation techniques. Theranostics, 789-804; Escudier. (2005). Vaccination of metastatic melanoma patients with autologous dendritic cell derived exosomes: results of the first phase I clinical trial. J transl. med, 1-13; Rood. (2010). Comparison of three methods for isolation of urinary microvesicles to identify biomarkers of nephrotic syndrome. Kidney Int, 810-816; and An M., W. J. (2018). Comparison of an optimized ultracentrifugation method versus size-exclusion chromatography for isolation of exosomes from human serum. J Proteome Res, 3599-3605).

Thus, a clear limitation to the clinical and practical use of exosomes is that methods of cost-effective purification, particularly in large quantities, exists along with a need for improved methods of exosome isolation, particularly those capable of scaling for industrial scale exosome preparation.

With that said, embodiments disclosed herein can provide methods and techniques of exosome isolation and/or purification, particularly milk exosomes, that can provide large-scale yields of exosomes. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### METHODS OF ISOLATING EXOSOMES

In recent years, it has become recognized that mammalian milk is enriched in exosomes and could offer a source for large scale production of these small extracellular vesicles. Bovine milk is produced in large quantities by the dairy industry, widely consumed, and generally immunologically well-tolerated by humans. Moreover, milk exosomes have been reported to cross from the gut into the blood circulation and traffic to various organs, including brain, heart, gut and lungs (Wolf T, B. S. (2015). The intestinal transport of bovine milk exosomes is mediated by endocytosis in human colon carcinoma Caco-2 cells and rat small intestinal IEC-6 cells. J Nutr, 2201-2206), which are properties that could provide a basis for oral administration of cargo (including but not limited to therapeutics) loaded exosomes. This being said, milk contains a diverse mixture of proteins, minerals, lipids, and other macromolecules. The complexity of the milk composition poses significant challenges to the purification of these extracellular vesicles. Casein proteins are a major constituent of milk, making up approximately 80% of all milk proteins. Caseins aggregate into large, colloidal complexes with calcium phosphate and other milk proteins to form what are referred to as casein micelles. These micelles are approximately 10 nm in diameter and can further coalesce into larger coagulated structures (Bhat, M. T. (2016). Casein Proteins: structural and functional aspects. Intech). Casein micelle aggregates are thought to bind to and ensnare exosomes via hydrostatic interactions, impeding separation from contaminating milk proteins; observations that are confirmed by transmission electron microscopy (TEM) analysis of milk-derived exosomal preparations (Sedykh S. E., B. E. (2020). Milk Exosomes: Isolation, Biochemistry, Morphology, and Perspectives of Use. In C. J. De Bona A. G., Extracellular Vesicles and their importance in human health. Intech Open). As a consequence, present methods for isolation of high-purity exosomes from milk are limited by contaminating proteins, such as casein. Described in several embodiments herein are methods that include incorporating chelation of Ca²⁺ and other divalent cations at specified temperatures that result in high-yield separation of structurally and functionally intact exosomes from milk proteins. In some embodiments, the casein micelle solubilization steps can be included in a method with UC-based and/or TFF and SEC filtration steps for exosome isolation thereby providing a basis for large-scale production of purified high quality exosomes from milk.

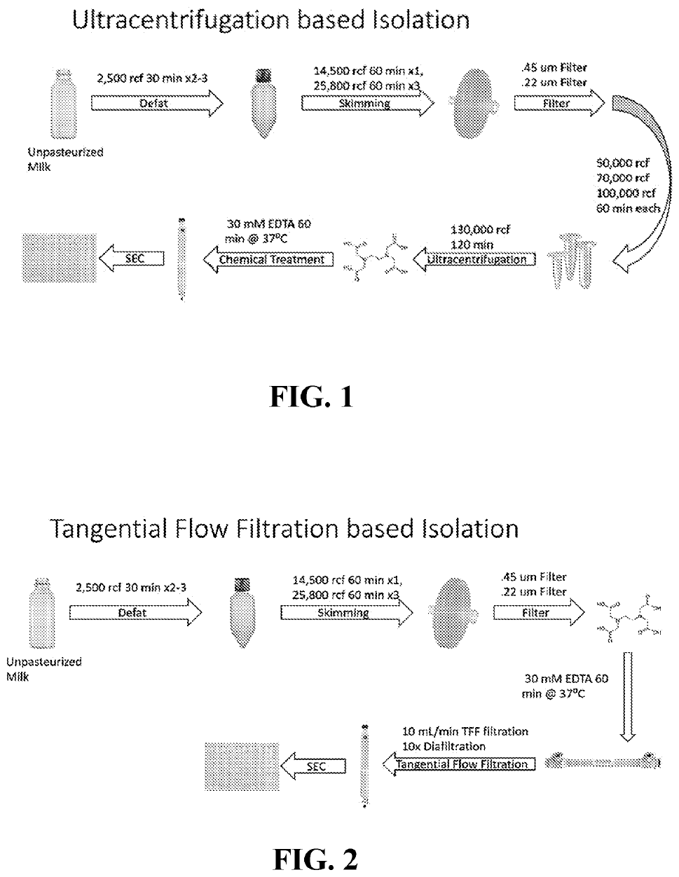

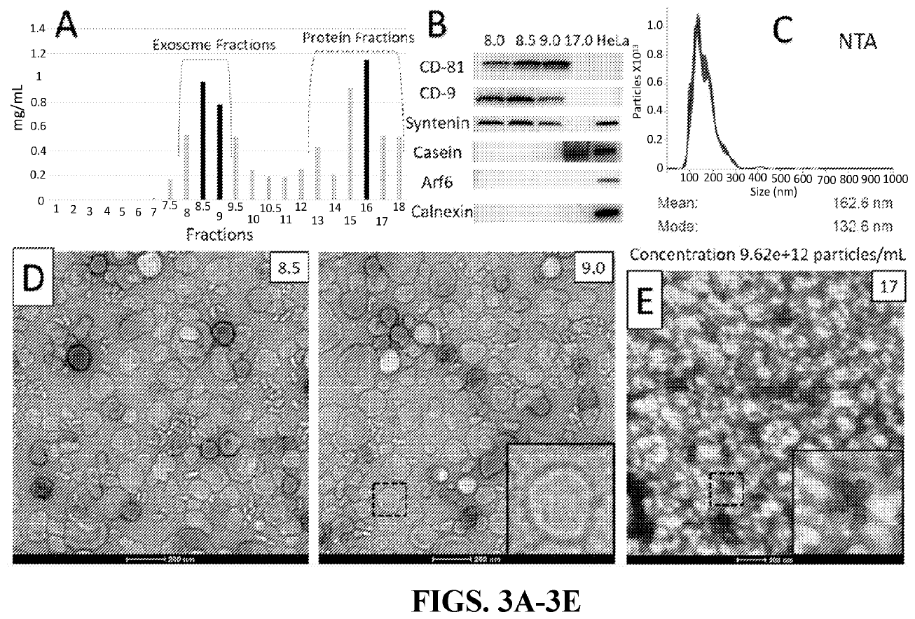

Described in certain example embodiments are methods of isolating exosomes from a biological fluid that includes (a) centrifuging a biological fluid under conditions suitable to separate fats from one or more other components of the biological fluid; (b) removing the separated fats from the biological fluid; (c) after step (b) centrifuging the remaining biological fluid one or more times and skimming any noticeable separated fats after each centrifuging in step (c); (d) filtering the remaining biological fluid after step (c); (e) optionally performing one or more ultracentrifugation steps after (d); (f) chelating divalent cations with ethylenediaminetetraacetic acid (EDTA) and/or other chelator(s), including, but not limited to, 1,2-bis(o-aminophenoxy)ethane-N,N,N′,N′-tetraacetic acid (BAPTA), ethylene glycol-bis((3-aminoethyl ether)-N,N,N′,N′-tetraacetic acid (EGTA), sodium citrate, and nitrophen at equivalent chelation concentrations, at about 30-42 degrees Celsius after (d) or optionally (e) and optionally for about 15-120 minutes; and (g) after (f), optionally performing tangential flow filtration to obtain a retentate, wherein the retentate is optionally ultracentrifuged via one or more ultracentrifugation steps or stored at −80 degrees C., and optionally fractionating the retentate, optionally via column separation, after the retentate is optionally ultracentrifuged or stored at −80 degrees C., wherein the method comprises step (e) or step (g) but not both. See e.g., FIGS. 1-2.

In some embodiments, dialysis can be used in the method to reduce ion concentrations. In some embodiments, the chelator is a divalent cation chelator. In some embodiments, the chelator is EDTA and/or other chelator, including but not limited to, BAPDTA, EGTA, sodium citrate, nitrophen which are included at equivalent chelation concentrations. In some embodiments, the concentration of chelator can range from about 10 mM to about 100 mM. In some embodiments, the chelator is EDTA and is present at about 30 mM. In some embodiments, chelating divalent cations occurs at about 37 degrees Celsius. In some embodiments, chelation of divalent cations occurs at about 37 degrees Celsius and is carried out for about 60 minutes.

In certain example embodiments are methods of isolating exosomes from a biological fluid that includes (a) centrifuging a biological fluid under conditions suitable to separate fats from one or more other components of the biological fluid; (b) removing the separated fats from the biological fluid; (c) after step (b) centrifuging the remaining biological fluid one or more times and skimming any noticeable separated fats after each centrifuging in step (c); (d) filtering the remaining biological fluid after step (c); (e) optionally performing one or more ultracentrifugation steps after (d); (0 chelating divalent cations with EDTA at about 30 mM at about 30-42 degrees Celsius after (d) or optionally (e) and optionally for about 15-120 minutes; and (g) after (f), optionally performing tangential flow filtration to obtain a retentate, wherein the retentate is optionally ultracentrifuged via one or more ultracentrifugation steps or stored at −80 degrees C., and optionally fractionating the retentate, optionally via column separation, after the retentate is optionally ultracentrifuged or stored at −80 degrees C., wherein the method comprises step (e) or step (g) but not both.

In certain example embodiments are methods of isolating exosomes from a biological fluid that includes (a) centrifuging a biological fluid under conditions suitable to separate fats from one or more other components of the biological fluid; (b) removing the separated fats from the biological fluid; (c) after step (b) centrifuging the remaining biological fluid one or more times and skimming any noticeable separated fats after each centrifuging in step (c); (d) filtering the remaining biological fluid after step (c); (e) optionally performing one or more ultracentrifugation steps after (d); (f) chelating divalent cations with EDTA at about 30 mM at about 37 degrees Celsius optionally for about 60 minutes after (d) or optionally (e); and (g) after (f), optionally performing tangential flow filtration to obtain a retentate, wherein the retentate is optionally ultracentrifuged via one or more ultracentrifugation steps or stored at −80 degrees C., and optionally fractionating the retentate, optionally via column separation, after the retentate is optionally ultracentrifuged or stored at −80 degrees C., wherein the method comprises step (e) or step (g) but not both.

In some embodiments, (f) is performed at about 30 degrees C., 30.5 degrees C., 31 degrees C., 31.5 degrees C., 32 degrees C., 32.5 degrees C., 33 degrees C., 33.5 degrees C., 34 degrees C., 34.5 degrees C., 35 degrees C., 35.5 degrees C., 36 degrees C., 36.5 degrees C., 37degrees C., 37.5 degrees C., 38 degrees C., 38.5 degrees C., 39 degrees C., 39.5 degrees C., 40 degrees C., 40.5 degrees C., 41 degrees C., 41.5 degrees C., or about 42 degrees C.

In some embodiments, (f) is performed for about 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, 121 minutes, 122 minutes, 123 minutes, 124 minutes, 125 minutes, 126 minutes, 127 minutes, 128 minutes, 129 minutes, 130 minutes, 131 minutes, 132 minutes, 133 minutes, 134 minutes, 135 minutes, 136 minutes, 137 minutes, 138 minutes, 139 minutes, 140 minutes, 141 minutes, 142 minutes, 143 minutes, 144 minutes, 145 minutes, 146 minutes, 147 minutes, 148 minutes, 149 minutes, 150 minutes, 151 minutes, 152 minutes, 153 minutes, 154 minutes, 155 minutes, 156 minutes, 157 minutes, 158 minutes, 159 minutes, 160 minutes, 161 minutes, 162 minutes, 163 minutes, 164 minutes, 165 minutes, 166 minutes, 167 minutes, 168 minutes, 169 minutes, 170 minutes, 171 minutes, 172 minutes, 173 minutes, 174 minutes, 175 minutes, 176 minutes, 177 minutes, 178 minutes, 179 minutes, or to about 180 minutes.

In some embodiments, the concentration of the chelator, such as EDTA, BAPDTA, EGTA, sodium citrate, nitrohpen, and/or the like is about 0 mM, 10.5 mM, 11 mM, 11.5 mM, 12 mM, 12.5 mM, 13 mM, 13.5 mM, 14 mM, 14.5 mM, 15 mM, 15.5 mM, 16 mM, 16.5 mM, 17 mM, 17.5 mM, 18 mM, 18.5 mM, 19 mM, 19.5 mM, 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, 38.5 mM, 39 mM, 39.5 mM, 40 mM, 40.5 mM, 41 mM, 41.5 mM, 42 mM, 42.5 mM, 43 mM, 43.5 mM, 44 mM, 44.5 mM, 45 mM, 45.5 mM, 46 mM, 46.5 mM, 47 mM, 47.5 mM, 48 mM, 48.5 mM, 49 mM, 49.5 mM, 50 mM, 50.5 mM, 51 mM, 51.5 mM, 52 mM, 52.5 mM, 53 mM, 53.5 mM, 54 mM, 54.5 mM, 55 mM, 55.5 mM, 56 mM, 56.5 mM, 57 mM, 57.5 mM, 58 mM, 58.5 mM, 59 mM, 59.5 mM, 60 mM, 60.5 mM, 61 mM, 61.5 mM, 62 mM, 62.5 mM, 63 mM, 63.5 mM, 64 mM, 64.5 mM, 65 mM, 65.5 mM, 66 mM, 66.5 mM, 67 mM, 67.5 mM, 68 mM, 68.5 mM, 69 mM, 69.5 mM, 70 mM, 70.5 mM, 71 mM, 71.5 mM, 72 mM, 72.5 mM, 73 mM, 73.5 mM, 74 mM, 74.5 mM, 75 mM, 75.5 mM, 76 mM, 76.5 mM, 77 mM, 77.5 mM, 78 mM, 78.5 mM, 79 mM, 79.5 mM, 80 mM, 80.5 mM, 81 mM, 81.5 mM, 82 mM, 82.5 mM, 83 mM, 83.5 mM, 84 mM, 84.5 mM, 85 mM, 85.5 mM, 86 mM, 86.5 mM, 87 mM, 87.5 mM, 88 mM, 88.5 mM, 89 mM, 89.5 mM, 90 mM, 90.5 mM, 91 mM, 91.5 mM, 92 mM, 92.5 mM, 93 mM, 93.5 mM, 94 mM, 94.5 mM, 95 mM, 95.5 mM, 96 mM, 96.5 mM, 97 mM, 97.5 mM, 98 mM, 98.5 mM, 99 mM, 99.5 mM, to/or about 100 mM.

In certain example embodiments, the biological fluids contains caseins. In certain example embodiments, the biological fluid is mammalian milk. In certain example embodiments, the biological fluid is unpasteurized. In some embodiments, the mammalian milk is unpasteurized. In some embodiments, the mammalian milk is pasteurized. In some embodiments, the mammalian milk is bovine milk, ovine milk, porcine milk, camelid milk, equine milk, capra milk, human milk, and/or the like.

In certain example embodiments, steps (a) and (b) together are repeated 1-5 times. In some embodiments, steps (a) and (b) together are repeated 1, 2, 3, 4, or 5 times.

In certain example embodiments, step (a), (b), (c), (d), (e), (g), or any combination thereof is performed at or at about 4 degrees Celsius.

In certain example embodiments, (a) comprises centrifuging the biological fluid at about 2,500 rcf. In certain example embodiment, (a) comprises centrifuging the biological fluid at about 2,000 rcf to about 3,000 rcf. In certain example embodiment, (a) comprises centrifuging the biological fluid at about 2000 rcf, 2010 rcf, 2020 rcf, 2030 rcf, 2040 rcf, 2050 rcf, 2060 rcf, 2070 rcf, 2080 rcf, 2090 rcf, 2100 rcf, 2110 rcf, 2120 rcf, 2130 rcf, 2140 rcf, 2150rcf, 2160 rcf, 2170 rcf, 2180 rcf, 2190 rcf, 2200 rcf, 2210 rcf, 2220 rcf, 2230 rcf, 2240 rcf, 2250 rcf, 2260 rcf, 2270 rcf, 2280 rcf, 2290 rcf, 2300 rcf, 2310 rcf, 2320 rcf, 2330 rcf, 2340 rcf, 2350 rcf, 2360 rcf, 2370 rcf, 2380 rcf, 2390 rcf, 2400 rcf, 2410 rcf, 2420 rcf, 2430 rcf, 2440 rcf, 2450 rcf, 2460 rcf, 2470 rcf, 2480 rcf, 2490 rcf, 2500 rcf, 2510 rcf, 2520 rcf, 2530 rcf, 2540 rcf, 2550 rcf, 2560 rcf, 2570 rcf, 2580 rcf, 2590 rcf, 2600 rcf, 2610 rcf, 2620 rcf, 2630 rcf, 2640 rcf, 2650 rcf, 2660 rcf, 2670 rcf, 2680 rcf, 2690 rcf, 2700 rcf, 2710 rcf, 2720 rcf, 2730 rcf, 2740 rcf, 2750 rcf, 2760 rcf, 2770 rcf, 2780 rcf, 2790 rcf, 2800 rcf, 2810 rcf, 2820 rcf, 2830 rcf, 2840 rcf, 2850 rcf, 2860 rcf, 2870 rcf, 2880 rcf, 2890 rcf, 2900 rcf, 2910 rcf, 2920 rcf, 2930 rcf, 2940 rcf, 2950 rcf, 2960 rcf, 2970 rcf, 2980 rcf, 2990 rcf, to/or about 3000 rcf.

In certain example embodiments, step (a) is repeated 1-3 times. In some embodiments, step (a) is repeated 1, 2, or 3 times.

In certain example embodiments, (b) includes a first centrifugation followed by a second centrifugation. In certain example embodiments, the first centrifugation includes centrifuging the remaining biological fluid at about 14,500 rcf for about 60 minutes. In certain example embodiments, the first centrifugation includes centrifuging the remaining biological fluid at about 13,500 rcf to about 15,500 rcf for about 45 to about 75 minutes. In some embodiments, the first centrifugation includes centrifuging the remaining biological fluid at about 13500 rcf, 13550 rcf, 13600 rcf, 13650 rcf, 13700 rcf, 13750 rcf, 13800 rcf, 13850 rcf, 13900 rcf, 13950 rcf, 14000 rcf, 14050 rcf, 14100 rcf, 14150 rcf, 14200 rcf, 14250 rcf, 14300 rcf, 14350 rcf, 14400 rcf, 14450 rcf, 14500 rcf, 14550 rcf, 14600 rcf, 14650 rcf, 14700 rcf, 14750 rcf, 14800 rcf, 14850 rcf, 14900 rcf, 14950 rcf, 15000 rcf, 15050 rcf, 15100 rcf, 15150 rcf, 15200 rcf, 15250 rcf, 15300 rcf, 15350 rcf, 15400 rcf, 15450 rcf, or at about 15500 rcf for about 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, or about 75 min.

In certain example embodiments, the second centrifugation is performed on the biological fluid remaining after the first centrifugation and wherein the second centrifugation is performed at about 25,800 rcf for about 60 minutes. In certain example embodiments, the second centrifugation is performed on the biological fluid remaining after the first centrifugation and the second centrifugation is performed at about 24,800 to about 26,800 rcf for about 45 to about 75 minutes. In certain example embodiments, the second centrifugation is performed on the biological fluid remaining after the first centrifugation and the second centrifugation is performed at about 24800 rcf, 24850 rcf, 24900 rcf, 24950 rcf, 25000 rcf, 25050 rcf, 25100 rcf, 25150 rcf, 25200 rcf, 25250 rcf, 25300 rcf, 25350 rcf, 25400 rcf, 25450 rcf, 25500 rcf, 25550 rcf, 25600 rcf, 25650 rcf, 25700 rcf, 25750 rcf, 25800 rcf, 25850 rcf, 25900 rcf, 25950 rcf, 26000 rcf, 26050 rcf, 26100 rcf, 26150 rcf, 26200 rcf, 26250 rcf, 26300 rcf, 26350 rcf, 26400 rcf, 26450 rcf, 26500 rcf, 26550 rcf, 26600 rcf, 26650 rcf, 26700 rcf, 26750 rcf, 26800 rcf for about 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, or about 75 min.

In certain example embodiments, the second centrifugation is repeated 1-3 times with each repetition being performed on the remaining biological fluid from the centrifugation immediately prior. In certain example embodiments, the second centrifugation is repeated 1, 2, or 3 times with each repetition being performed on the remaining biological fluid from the centrifugation immediately prior

In certain example embodiments, (d) includes filtering the remaining biological fluid through one or more filters in series ranging from about a 0.45 micron filter to about a 0.22 micron filter. In some embodiments, each filter in the series is independently selected from a 0.22 micron, 0.23 micron, 0.24 micron, 0.25 micron, 0.26 micron, 0.27 micron, 0.28 micron, 0.29 micron, 0.3 micron, 0.31 micron, 0.32 micron, 0.33 micron, 0.34 micron, 0.35 micron, 0.36 micron, 0.37 micron, 0.38 micron, 0.39 micron, 0.4 micron, 0.41 micron, 0.42 micron, 0.43 micron, 0.44 micron, or 0.45 micron filter. In some embodiments, the number of filters in series ranges 1-10, such as 1, 2, 3, 4, 5, 6, 7, 8, 9, or 10 filters in series. In some embodiments, all filters in the series are the same size cut off. In some embodiments, at least 2 filters in the series have the same size cut off. In some embodiments, at least 2 filters in the series have different size cut offs. In some embodiments, all the filters in the series have different size cut offs. In some embodiments, the size exclusion decrease from large to small along a series of filters. For example, in a series of 3 filters, the first filter can be a 0.45 micron filter, the second filter can be a 0.3 micron filter and the last filter can be a 0.22 filter. Other configurations of filters in series will be appreciated in view of the description herein. In some embodiments, the filters in series are all the same material. In some embodiments, the filters in series are all different materials. In some embodiments, at least 2 filters in the series are the same material. In some embodiments, at least 2 filters are made of different materials. Exemplary filters include, but are not limited to, membrane filters (e.g., polyethersulfone membrane filters, polyvinylidene fluoride membrane filters, cellulose membrane filters, mixed cellulose esters membrane filters, cellulose acetate membrane filters, cellulose nitrate membrane filters, polyamide membrane filters, polycarbonate membrane filters, polytetrafluoroethylene membrane filters, polypropylene membrane filters, nitrocellulose membrane filters, and/or the like), glass fiber or bead filters, and/or the like.

In certain example embodiments, (d) includes filtering the remaining biological fluid through an about 0.45 micron filter followed by filtering the remaining biological fluid through an about 0.22 micron filter.

In certain example embodiments, (e) includes 2 or more serial ultracentrifugation steps, wherein each step is performed on the remaining biological fluid from the prior ultracentrifugation. In certain example embodiments, (e) includes 2-10 (e.g., 2, 3, 4, 5, 6, 7, 8, 9, or 10) serial ultracentrifugation steps, wherein each step is performed on the remaining biological fluid from the prior ultracentrifugation. In certain example embodiments, (e) includes an ultracentrifugation step performed at about 50,000 rcf, an ultracentrifugation step performed at about 70,000 rcf, an ultracentrifugation step performed at about 100,000 rcf, or any combination thereof. In certain example embodiments, (e) includes an ultracentrifugation step performed at about 45,000 to about 55,000 rcf, an ultracentrifugation step performed at about 65,000 to about 75,000 rcf, an ultracentrifugation step performed at about 90,000 to about 110,000 rcf, or any combination thereof. In certain example embodiments, (e) includes an ultracentrifugation step performed at about 45,000 to about 55,000 rcf (e.g., at about 45000 rcf, 45100 rcf, 45200 rcf, 45300 rcf, 45400 rcf, 45500 rcf, 45600 rcf, 45700 rcf, 45800 rcf, 45900 rcf, 46000 rcf, 46100 rcf, 46200 rcf, 46300 rcf, 46400 rcf, 46500 rcf, 46600 rcf, 46700 rcf, 46800 rcf, 46900 rcf, 47000 rcf, 47100 rcf, 47200 rcf, 47300 rcf, 47400 rcf, 47500 rcf, 47600 rcf, 47700 rcf, 47800 rcf, 47900 rcf, 48000 rcf, 48100 rcf, 48200 rcf, 48300 rcf, 48400 rcf, 48500 rcf, 48600 rcf, 48700 rcf, 48800 rcf, 48900 rcf, 49000 rcf, 49100 rcf, 49200 rcf, 49300 rcf, 49400 rcf, 49500 rcf, 49600 rcf, 49700 rcf, 49800 rcf, 49900 rcf, 50000 rcf, 50100 rcf, 50200 rcf, 50300 rcf, 50400 rcf, 50500 rcf, 50600 rcf, 50700 rcf, 50800 rcf, 50900 rcf, 51000 rcf, 51100 rcf, 51200 rcf, 51300 rcf, 51400 rcf, 51500 rcf, 51600 rcf, 51700 rcf, 51800 rcf, 51900 rcf, 52000 rcf, 52100 rcf, 52200 rcf, 52300 rcf, 52400 rcf, 52500 rcf, 52600 rcf, 52700 rcf, 52800 rcf, 52900 rcf, 53000 rcf, 53100 rcf, 53200 rcf, 53300 rcf, 53400 rcf, 53500 rcf, 53600 rcf, 53700 rcf, 53800 rcf, 53900 rcf, 54000 rcf, 54100 rcf, 54200 rcf, 54300 rcf, 54400 rcf, 54500 rcf, 54600 rcf, 54700 rcf, 54800 rcf, 54900 rcf, or at about 55000 rcf), an ultracentrifugation step performed at about 65,000 to about 75,000 rcf (e.g., at about 65000 rcf, 65100 rcf, 65200 rcf, 65300 rcf, 65400 rcf, 65500 rcf, 65600 rcf, 65700 rcf, 65800 rcf, 65900 rcf, 66000 rcf, 66100 rcf, 66200 rcf, 66300 rcf, 66400 rcf, 66500 rcf, 66600 rcf, 66700 rcf, 66800 rcf, 66900 rcf, 67000 rcf, 67100 rcf, 67200 rcf, 67300 rcf, 67400 rcf, 67500 rcf, 67600 rcf, 67700 rcf, 67800 rcf, 67900 rcf, 68000 rcf, 68100 rcf, 68200 rcf, 68300 rcf, 68400 rcf, 68500 rcf, 68600 rcf, 68700 rcf, 68800 rcf, 68900 rcf, 69000 rcf, 69100 rcf, 69200 rcf, 69300 rcf, 69400 rcf, 69500 rcf, 69600 rcf, 69700 rcf, 69800 rcf, 69900 rcf, 70000 rcf, 70100 rcf, 70200 rcf, 70300 rcf, 70400 rcf, 70500 rcf, 70600 rcf, 70700 rcf, 70800 rcf, 70900 rcf, 71000 rcf, 71100 rcf, 71200 rcf, 71300 rcf, 71400 rcf, 71500 rcf, 71600 rcf, 71700 rcf, 71800 rcf, 71900 rcf, 72000 rcf, 72100 rcf, 72200 rcf, 72300 rcf, 72400 rcf, 72500 rcf, 72600 rcf, 72700 rcf, 72800 rcf, 72900 rcf, 73000 rcf, 73100 rcf, 73200 rcf, 73300 rcf, 73400 rcf, 73500 rcf, 73600 rcf, 73700 rcf, 73800 rcf, 73900 rcf, 74000 rcf, 74100 rcf, 74200 rcf, 74300 rcf, 74400 rcf, 74500 rcf, 74600 rcf, 74700 rcf, 74800 rcf, 74900 rcf, or at about 75000 rcf), an ultracentrifugation step performed at about 90,000 to about 110,000 rcf (e.g., 90000 rcf, 90100 rcf, 90200 rcf, 90300 rcf, 90400 rcf, 90500 rcf, 90600 rcf, 90700 rcf, 90800 rcf, 90900 rcf, 91000 rcf, 91100 rcf, 91200 rcf, 91300 rcf, 91400 rcf, 91500 rcf, 91600 rcf, 91700 rcf, 91800 rcf, 91900 rcf, 92000 rcf, 92100 rcf, 92200 rcf, 92300 rcf, 92400 rcf, 92500 rcf, 92600 rcf, 92700 rcf, 92800 rcf, 92900 rcf, 93000 rcf, 93100 rcf, 93200 rcf, 93300 rcf, 93400 rcf, 93500 rcf, 93600 rcf, 93700 rcf, 93800 rcf, 93900 rcf, 94000 rcf, 94100 rcf, 94200 rcf, 94300 rcf, 94400 rcf, 94500 rcf, 94600 rcf, 94700 rcf, 94800 rcf, 94900 rcf, 95000 rcf, 95100 rcf, 95200 rcf, 95300 rcf, 95400 rcf, 95500 rcf, 95600 rcf, 95700 rcf, 95800 rcf, 95900 rcf, 96000 rcf, 96100 rcf, 96200 rcf, 96300 rcf, 96400 rcf, 96500 rcf, 96600 rcf, 96700 rcf, 96800 rcf, 96900 rcf, 97000 rcf, 97100 rcf, 97200 rcf, 97300 rcf, 97400 rcf, 97500 rcf, 97600 rcf, 97700 rcf, 97800 rcf, 97900 rcf, 98000 rcf, 98100 rcf, 98200 rcf, 98300 rcf, 98400 rcf, 98500 rcf, 98600 rcf, 98700 rcf, 98800 rcf, 98900 rcf, 99000 rcf, 99100 rcf, 99200 rcf, 99300 rcf, 99400 rcf, 99500 rcf, 99600 rcf, 99700 rcf, 99800 rcf, 99900 rcf, 100000 rcf, 100100 rcf, 100200 rcf, 100300 rcf, 100400 rcf, 100500 rcf, 100600 rcf, 100700 rcf, 100800 rcf, 100900 rcf, 101000 rcf, 101100 rcf, 101200 rcf, 101300 rcf, 101400 rcf, 101500 rcf, 101600 rcf, 101700 rcf, 101800 rcf, 101900 rcf, 102000 rcf, 102100 rcf, 102200 rcf, 102300 rcf, 102400 rcf, 102500 rcf, 102600 rcf, 102700 rcf, 102800 rcf, 102900 rcf, 103000 rcf, 103100 rcf, 103200 rcf, 103300 rcf, 103400 rcf, 103500 rcf, 103600 rcf, 103700 rcf, 103800 rcf, 103900 rcf, 104000 rcf, 104100 rcf, 104200 rcf, 104300 rcf, 104400 rcf, 104500 rcf, 104600 rcf, 104700 rcf, 104800 rcf, 104900 rcf, 105000 rcf, 105100 rcf, 105200 rcf, 105300 rcf, 105400 rcf, 105500 rcf, 105600 rcf, 105700 rcf, 105800 rcf, 105900 rcf, 106000 rcf, 106100 rcf, 106200 rcf, 106300 rcf, 106400 rcf, 106500 rcf, 106600 rcf, 106700 rcf, 106800 rcf, 106900 rcf, 107000 rcf, 107100 rcf, 107200 rcf, 107300 rcf, 107400 rcf, 107500 rcf, 107600 rcf, 107700 rcf, 107800 rcf, 107900 rcf, 108000 rcf, 108100 rcf, 108200 rcf, 108300 rcf, 108400 rcf, 108500 rcf, 108600 rcf, 108700 rcf, 108800 rcf, 108900 rcf, 109000 rcf, 109100 rcf, 109200 rcf, 109300 rcf, 109400 rcf, 109500 rcf, 109600 rcf, 109700 rcf, 109800 rcf, 109900 rcf, or at about 110000 rcf), or any combination thereof. In certain example embodiments, the one or more of the one or more ultracentrifugation steps are each performed for about 60 minutes. In certain example embodiments, the one or more of the one or more ultracentrifugation steps are each performed for about 45-75 minutes. In certain example embodiments, the one or more of the one or more ultracentrifugation steps are each performed for about 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, or about 75 min.

In certain example embodiments, (e) comprises a final ultracentrifugation step performed at about 130,000 rcf for about 120 minutes, the resulting fluid is discarded, and the remaining pellet is resuspended in a suitable volume of a suitable solution prior to (f). In certain example embodiments, (e) comprises a final ultracentrifugation step performed at about 115,000 to about 145,000 rcf for about 90-150 minutes, the resulting fluid is discarded, and the remaining pellet is resuspended in a suitable volume of a suitable solution prior to (f). In certain example embodiments, (e) comprises a final ultracentrifugation step performed at about 115,000 to about 145,000 rcf (e.g., 115000 rcf, 115100 rcf, 115200 rcf, 115300 rcf, 115400 rcf, 115500 rcf, 115600 rcf, 115700 rcf, 115800 rcf, 115900 rcf, 116000 rcf, 116100 rcf, 116200 rcf, 116300 rcf, 116400 rcf, 116500 rcf, 116600 rcf, 116700 rcf, 116800 rcf, 116900 rcf, 117000 rcf, 117100 rcf, 117200 rcf, 117300 rcf, 117400 rcf, 117500 rcf, 117600 rcf, 117700 rcf, 117800 rcf, 117900 rcf, 118000 rcf, 118100 rcf, 118200 rcf, 118300 rcf, 118400 rcf, 118500 rcf, 118600 rcf, 118700 rcf, 118800 rcf, 118900 rcf, 119000 rcf, 119100 rcf, 119200 rcf, 119300 rcf, 119400 rcf, 119500 rcf, 119600 rcf, 119700 rcf, 119800 rcf, 119900 rcf, 120000 rcf, 120100 rcf, 120200 rcf, 120300 rcf, 120400 rcf, 120500 rcf, 120600 rcf, 120700 rcf, 120800 rcf, 120900 rcf, 121000 rcf, 121100 rcf, 121200 rcf, 121300 rcf, 121400rcf, 121500 rcf, 121600 rcf, 121700 rcf, 121800 rcf, 121900 rcf, 122000 rcf, 122100 rcf, 122200 rcf, 122300 rcf, 122400 rcf, 122500 rcf, 122600 rcf, 122700 rcf, 122800 rcf, 122900 rcf, 123000 rcf, 123100 rcf, 123200 rcf, 123300 rcf, 123400 rcf, 123500 rcf, 123600 rcf, 123700 rcf, 123800 rcf, 123900 rcf, 124000 rcf, 124100 rcf, 124200 rcf, 124300 rcf, 124400 rcf, 124500 rcf, 124600 rcf, 124700 rcf, 124800 rcf, 124900 rcf, 125000 rcf, 125100 rcf, 125200 rcf, 125300 rcf, 125400 rcf, 125500 rcf, 125600 rcf, 125700 rcf, 125800 rcf, 125900 rcf, 126000 rcf, 126100 rcf, 126200 rcf, 126300 rcf, 126400 rcf, 126500 rcf, 126600 rcf, 126700 rcf, 126800 rcf, 126900 rcf, 127000 rcf, 127100 rcf, 127200 rcf, 127300 rcf, 127400 rcf, 127500 rcf, 127600 rcf, 127700 rcf, 127800 rcf, 127900 rcf, 128000 rcf, 128100 rcf, 128200 rcf, 128300 rcf, 128400 rcf, 128500 rcf, 128600 rcf, 128700 rcf, 128800 rcf, 128900 rcf, 129000 rcf, 129100 rcf, 129200 rcf, 129300 rcf, 129400 rcf, 129500 rcf, 129600 rcf, 129700 rcf, 129800 rcf, 129900 rcf, 130000 rcf, 130100 rcf, 130200 rcf, 130300 rcf, 130400 rcf, 130500 rcf, 130600 rcf, 130700 rcf, 130800 rcf, 130900 rcf, 131000 rcf, 131100 rcf, 131200 rcf, 131300 rcf, 131400 rcf, 131500 rcf, 131600 rcf, 131700 rcf, 131800 rcf, 131900 rcf, 132000 rcf, 132100 rcf, 132200 rcf, 132300 rcf, 132400 rcf, 132500 rcf, 132600 rcf, 132700 rcf, 132800 rcf, 132900 rcf, 133000 rcf, 133100 rcf, 133200 rcf, 133300 rcf, 133400 rcf, 133500 rcf, 133600 rcf, 133700 rcf, 133800 rcf, 133900 rcf, 134000 rcf, 134100 rcf, 134200 rcf, 134300 rcf, 134400 rcf, 134500 rcf, 134600 rcf, 134700 rcf, 134800 rcf, 134900 rcf, 135000 rcf, 135100 rcf, 135200 rcf, 135300 rcf, 135400 rcf, 135500 rcf, 135600 rcf, 135700 rcf, 135800 rcf, 135900 rcf, 136000 rcf, 136100 rcf, 136200 rcf, 136300 rcf, 136400 rcf, 136500 rcf, 136600 rcf, 136700 rcf, 136800 rcf, 136900 rcf, 137000 rcf, 137100 rcf, 137200 rcf, 137300 rcf, 137400 rcf, 137500 rcf, 137600 rcf, 137700 rcf, 137800 rcf, 137900 rcf, 138000 rcf, 138100 rcf, 138200 rcf, 138300 rcf, 138400 rcf, 138500 rcf, 138600 rcf, 138700 rcf, 138800 rcf, 138900 rcf, 139000 rcf, 139100 rcf, 139200 rcf, 139300 rcf, 139400 rcf, 139500 rcf, 139600 rcf, 139700 rcf, 139800 rcf, 139900 rcf, 140000 rcf, 140100 rcf, 140200 rcf, 140300 rcf, 140400 rcf, 140500 rcf, 140600 rcf, 140700 rcf, 140800 rcf, 140900 rcf, 141000 rcf, 141100 rcf, 141200 rcf, 141300 rcf, 141400 rcf, 141500 rcf, 141600 rcf, 141700 rcf, 141800 rcf, 141900 rcf, 142000 rcf, 142100 rcf, 142200 rcf, 142300 rcf, 142400 rcf, 142500 rcf, 142600 rcf, 142700 rcf, 142800 rcf, 142900 rcf, 143000 rcf, 143100 rcf, 143200 rcf, 143300 rcf, 143400 rcf, 143500 rcf, 143600 rcf, 143700 rcf, 143800 rcf, 143900 rcf, 144000 rcf, 144100 rcf, 144200 rcf, 144300 rcf, 144400 rcf, 144500 rcf, 144600 rcf, 144700 rcf, 144800 rcf, 144900 rcf, or at about 145000 rcf) for about 90-150 minutes (e.g., 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, 120 min, 121 min, 122 min, 123 min, 124 min, 125 min, 126 min, 127 min, 128 min, 129 min, 130 min, 131 min, 132 min, 133 min, 134 min, 135 min, 136 min, 137 min, 138 min, 139 min, 140 min, 141 min, 142 min, 143 min, 144 min, 145 min, 146 min, 147 min, 148 min, 149 min, or about 150 min), the resulting fluid is discarded, and the remaining pellet is resuspended in a suitable volume of a suitable solution prior to (f).

In certain example embodiments, the tangential flow filtration of (g) is performed using a 500 kDa ultrafiltration membrane. In certain example embodiments, the tangential flow filtration of (g) is performed using an ultrafiltration membrane with about a molecular weight cutoff ranging from about 250 kDa to about 750 kDa. In some embodiments the molecular weight cutoff of the ultrafiltration membrane with a molecular weight cutoff of about 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390kDa, 400kDa, 410kDa, 420kDa, 430kDa, 440kDa, 450kDa, 460kDa, 470kDa, 480kDa, 490kDa, 500kDa, 510kDa, 520kDa, 530kDa, 540kDa, 550kDa, 560kDa, 570kDa, 580kDa, 590kDa, 600kDa, 610kDa, 620kDa, 630kDa, 640kDa, 650kDa, 660kDa, 670kDa, 680kDa, 690kDa, 700kDa, 710kDa, 720kDa, 730kDa, 740kDa, or about 750kDa.

In certain example embodiments, the tangential flow filtration of (g) is performed at a flow rate of about 10 mL per minute. In certain example embodiments, the tangential flow filtration of (g) is performed at a flow rate ranging from about 5 mL to about 15 mL per minute. In certain example embodiments, the tangential flow filtration of (g) is performed at a flow rate of about 5 mL/min, 5.5 mL/min, 6 mL/min, 6.5 mL/min, 7 mL/min, 7.5 mL/min, 8 mL/min, 8.5 mL/min, 9 mL/min, 9.5 mL/min, 10 mL/min, 10.5 mL/min, 11 mL/min, 11.5 mL/min, 12 mL/min, 12.5 mL/min, 13 mL/min, 13.5 mL/min, 14 mL/min, 14.5 mL/min, or about 15 mL/min.

In certain example embodiments, in step (g), when the amount of remaining biological fluid reaches about ten percent of its starting volume before tangential flow filtration the retentate is diafiltered with a suitable buffer.

In certain example embodiments, the method further includes ultracentrifuging the retentate when the retentate reaches about 20 percent of the starting diafiltration amount. In certain example embodiments, the ultracentrifugation of the retentate is performed at about 130,000 rcf for about 120 at about 4 degrees Celsius. In certain example embodiments, the ultracentrifugation of the retentate performed at about 115,000 to about 145,000 rcf (e.g., 115000 rcf, 115050 rcf, 115100 rcf, 115150 rcf, 115200 rcf, 115250 rcf, 115300 rcf, 115350 rcf, 115400 rcf, 115450 rcf, 115500 rcf, 115550 rcf, 115600 rcf, 115650 rcf, 115700 rcf, 115750 rcf, 115800 rcf, 115850 rcf, 115900 rcf, 115950 rcf, 116000 rcf, 116050 rcf, 116100 rcf, 116150 rcf, 116200 rcf, 116250 rcf, 116300 rcf, 116350 rcf, 116400 rcf, 116450 rcf, 116500 rcf, 116550 rcf, 116600 rcf, 116650 rcf, 116700 rcf, 116750 rcf, 116800 rcf, 116850 rcf, 116900 rcf, 116950 rcf, 117000 rcf, 117050 rcf, 117100 rcf, 117150 rcf, 117200 rcf, 117250 rcf, 117300 rcf, 117350 rcf, 117400 rcf, 117450 rcf, 117500 rcf, 117550 rcf, 117600 rcf, 117650 rcf, 117700 rcf, 117750 rcf, 117800 rcf, 117850 rcf, 117900 rcf, 117950 rcf, 118000 rcf, 118050 rcf, 118100 rcf, 118150 rcf, 118200 rcf, 118250 rcf, 118300 rcf, 118350 rcf, 118400 rcf, 118450 rcf, 118500 rcf, 118550 rcf, 118600 rcf, 118650 rcf, 118700 rcf, 118750 rcf, 118800 rcf, 118850 rcf, 118900 rcf, 118950 rcf, 119000 rcf, 119050 rcf, 119100 rcf, 119150 rcf, 119200 rcf, 119250 rcf, 119300 rcf, 119350 rcf, 119400 rcf, 119450 rcf, 119500 rcf, 119550 rcf, 119600 rcf, 119650 rcf, 119700 rcf, 119750 rcf, 119800 rcf, 119850 rcf, 119900 rcf, 119950 rcf, 120000 rcf, 120050 rcf, 120100 rcf, 120150 rcf, 120200 rcf, 120250 rcf, 120300 rcf, 120350 rcf, 120400 rcf, 120450 rcf, 120500 rcf, 120550 rcf, 120600 rcf, 120650 rcf, 120700 rcf, 120750 rcf, 120800 rcf, 120850 rcf, 120900 rcf, 120950 rcf, 121000 rcf, 121050 rcf, 121100 rcf, 121150 rcf, 121200 rcf, 121250 rcf, 121300 rcf, 121350 rcf, 121400 rcf, 121450 rcf, 121500 rcf, 121550 rcf, 121600 rcf, 121650 rcf, 121700 rcf, 121750 rcf, 121800 rcf, 121850 rcf, 121900 rcf, 121950 rcf, 122000 rcf, 122050 rcf, 122100 rcf, 122150 rcf, 122200 rcf, 122250 rcf, 122300 rcf, 122350 rcf, 122400 rcf, 122450 rcf, 122500 rcf, 122550 rcf, 122600 rcf, 122650 rcf, 122700 rcf, 122750 rcf, 122800 rcf, 122850 rcf, 122900 rcf, 122950 rcf, 123000 rcf, 123050 rcf, 123100 rcf, 123150 rcf, 123200 rcf, 123250 rcf, 123300 rcf, 123350 rcf, 123400 rcf, 123450 rcf, 123500 rcf, 123550 rcf, 123600 rcf, 123650 rcf, 123700 rcf, 123750 rcf, 123800 rcf, 123850 rcf, 123900 rcf, 123950 rcf, 124000 rcf, 124050 rcf, 124100 rcf, 124150 rcf, 124200 rcf, 124250 rcf, 124300 rcf, 124350 rcf, 124400 rcf, 124450 rcf, 124500 rcf, 124550 rcf, 124600 rcf, 124650 rcf, 124700 rcf, 124750 rcf, 124800 rcf, 124850 rcf, 124900 rcf, 124950 rcf, 125000 rcf, 125050 rcf, 125100 rcf, 125150 rcf, 125200 rcf, 125250 rcf, 125300 rcf, 125350 rcf, 125400 rcf, 125450 rcf, 125500 rcf, 125550 rcf, 125600 rcf, 125650 rcf, 125700 rcf, 125750 rcf, 125800 rcf, 125850 rcf, 125900 rcf, 125950 rcf, 126000 rcf, 126050 rcf, 126100 rcf, 126150 rcf, 126200 rcf, 126250 rcf, 126300 rcf, 126350 rcf, 126400 rcf, 126450 rcf, 126500 rcf, 126550 rcf, 126600 rcf, 126650 rcf, 126700 rcf, 126750 rcf, 126800 rcf, 126850 rcf, 126900 rcf, 126950 rcf, 127000 rcf, 127050 rcf, 127100 rcf, 127150 rcf, 127200 rcf, 127250 rcf, 127300 rcf, 127350 rcf, 127400 rcf, 127450 rcf, 127500 rcf, 127550 rcf, 127600 rcf, 127650 rcf, 127700 rcf, 127750 rcf, 127800 rcf, 127850 rcf, 127900 rcf, 127950 rcf, 128000 rcf, 128050 rcf, 128100 rcf, 128150 rcf, 128200 rcf, 128250 rcf, 128300 rcf, 128350 rcf, 128400 rcf, 128450 rcf, 128500 rcf, 128550 rcf, 128600 rcf, 128650 rcf, 128700 rcf, 128750 rcf, 128800 rcf, 128850 rcf, 128900 rcf, 128950 rcf, 129000 rcf, 129050 rcf, 129100 rcf, 129150 rcf, 129200 rcf, 129250 rcf, 129300 rcf, 129350 rcf, 129400 rcf, 129450 rcf, 129500 rcf, 129550 rcf, 129600 rcf, 129650 rcf, 129700 rcf, 129750 rcf, 129800 rcf, 129850 rcf, 129900 rcf, 129950 rcf, 130000 rcf, 130050 rcf, 130100 rcf, 130150 rcf, 130200 rcf, 130250 rcf, 130300 rcf, 130350 rcf, 130400 rcf, 130450 rcf, 130500 rcf, 130550 rcf, 130600 rcf, 130650 rcf, 130700 rcf, 130750 rcf, 130800 rcf, 130850 rcf, 130900 rcf, 130950 rcf, 131000 rcf, 131050 rcf, 131100 rcf, 131150 rcf, 131200 rcf, 131250 rcf, 131300 rcf, 131350 rcf, 131400 rcf, 131450 rcf, 131500 rcf, 131550 rcf, 131600 rcf, 131650 rcf, 131700 rcf, 131750 rcf, 131800 rcf, 131850 rcf, 131900 rcf, 131950 rcf, 132000 rcf, 132050 rcf, 132100 rcf, 132150 rcf, 132200 rcf, 132250 rcf, 132300 rcf, 132350 rcf, 132400 rcf, 132450 rcf, 132500 rcf, 132550 rcf, 132600 rcf, 132650 rcf, 132700 rcf, 132750 rcf, 132800 rcf, 132850 rcf, 132900 rcf, 132950 rcf, 133000 rcf, 133050 rcf, 133100 rcf, 133150 rcf, 133200 rcf, 133250 rcf, 133300 rcf, 133350 rcf, 133400 rcf, 133450 rcf, 133500 rcf, 133550 rcf, 133600 rcf, 133650 rcf, 133700 rcf, 133750 rcf, 133800 rcf, 133850 rcf, 133900 rcf, 133950 rcf, 134000 rcf, 134050 rcf, 134100 rcf, 134150 rcf, 134200 rcf, 134250 rcf, 134300 rcf, 134350 rcf, 134400 rcf, 134450 rcf, 134500 rcf, 134550 rcf, 134600 rcf, 134650 rcf, 134700 rcf, 134750 rcf, 134800 rcf, 134850 rcf, 134900 rcf, 134950 rcf, 135000 rcf, 135050 rcf, 135100 rcf, 135150 rcf, 135200 rcf, 135250 rcf, 135300 rcf, 135350 rcf, 135400 rcf, 135450 rcf, 135500 rcf, 135550 rcf, 135600 rcf, 135650 rcf, 135700 rcf, 135750 rcf, 135800 rcf, 135850 rcf, 135900 rcf, 135950 rcf, 136000 rcf, 136050 rcf, 136100 rcf, 136150 rcf, 136200 rcf, 136250 rcf, 136300 rcf, 136350 rcf, 136400 rcf, 136450 rcf, 136500 rcf, 136550 rcf, 136600 rcf, 136650 rcf, 136700 rcf, 136750 rcf, 136800 rcf, 136850 rcf, 136900 rcf, 136950 rcf, 137000 rcf, 137050 rcf, 137100 rcf, 137150 rcf, 137200 rcf, 137250 rcf, 137300 rcf, 137350 rcf, 137400 rcf, 137450 rcf, 137500 rcf, 137550 rcf, 137600 rcf, 137650 rcf, 137700 rcf, 137750 rcf, 137800 rcf, 137850 rcf, 137900 rcf, 137950 rcf, 138000 rcf, 138050 rcf, 138100 rcf, 138150 rcf, 138200 rcf, 138250 rcf, 138300 rcf, 138350 rcf, 138400 rcf, 138450 rcf, 138500 rcf, 138550 rcf, 138600 rcf, 138650 rcf, 138700 rcf, 138750 rcf, 138800 rcf, 138850 rcf, 138900 rcf, 138950 rcf, 139000 rcf, 139050 rcf, 139100 rcf, 139150 rcf, 139200 rcf, 139250 rcf, 139300 rcf, 139350 rcf, 139400 rcf, 139450 rcf, 139500 rcf, 139550 rcf, 139600 rcf, 139650 rcf, 139700 rcf, 139750 rcf, 139800 rcf, 139850 rcf, 139900 rcf, 139950 rcf, 140000 rcf, 140050 rcf, 140100 rcf, 140150 rcf, 140200 rcf, 140250 rcf, 140300 rcf, 140350 rcf, 140400 rcf, 140450 rcf, 140500 rcf, 140550 rcf, 140600 rcf, 140650 rcf, 140700 rcf, 140750 rcf, 140800 rcf, 140850 rcf, 140900 rcf, 140950 rcf, 141000 rcf, 141050 rcf, 141100 rcf, 141150 rcf, 141200 rcf, 141250 rcf, 141300 rcf, 141350 rcf, 141400 rcf, 141450 rcf, 141500 rcf, 141550 rcf, 141600 rcf, 141650 rcf, 141700 rcf, 141750 rcf, 141800 rcf, 141850 rcf, 141900 rcf, 141950 rcf, 142000 rcf, 142050 rcf, 142100 rcf, 142150 rcf, 142200 rcf, 142250 rcf, 142300 rcf, 142350 rcf, 142400 rcf, 142450 rcf, 142500 rcf, 142550 rcf, 142600 rcf, 142650 rcf, 142700 rcf, 142750 rcf, 142800 rcf, 142850 rcf, 142900 rcf, 142950 rcf, 143000 rcf, 143050 rcf, 143100 rcf, 143150 rcf, 143200 rcf, 143250 rcf, 143300 rcf, 143350 rcf, 143400 rcf, 143450 rcf, 143500 rcf, 143550 rcf, 143600 rcf, 143650 rcf, 143700 rcf, 143750 rcf, 143800 rcf, 143850 rcf, 143900 rcf, 143950 rcf, 144000 rcf, 144050 rcf, 144100 rcf, 144150 rcf, 144200 rcf, 144250 rcf, 144300 rcf, 144350 rcf, 144400 rcf, 144450 rcf, 144500 rcf, 144550 rcf, 144600 rcf, 144650 rcf, 144700 rcf, 144750 rcf, 144800 rcf, 144850 rcf, 144900 rcf, 144950 rcf, or about145000 rcf) for about 90 minutes to about 150 minutes e.g., (about 90 min, 91 min, 92 min, 93 min, 94 min, 95 min, 96 min, 97 min, 98 min, 99 min, 100 min, 101 min, 102 min, 103 min, 104 min, 105 min, 106 min, 107 min, 108 min, 109 min, 110 min, 111 min, 112 min, 113 min, 114 min, 115 min, 116 min, 117 min, 118 min, 119 min, 120 min, 121 min, 122 min, 123 min, 124 min, 125 min, 126 min, 127 min, 128 min, 129 min, 130 min, 131 min, 132 min, 133 min, 134 min, 135 min, 136 min, 137 min, 138 min, 139 min, 140 min, 141 min, 142 min, 143 min, 144 min, 145 min, 146 min, 147 min, 148 min, 149 min, or about 150 min) at about 4 degrees Celsius.

In certain example embodiments, the retentate is not ultracentrifuged prior to optionally fractionating the retentate. In these embodiments, the retentate is stored at about −80 degrees C. prior to fractionation that is optionally performed via column separation.

Optional fractionating of the retentate can be performed via any suitable method, including but not limited to column separation (based on size, charge, affinity, avidity, or other method or separation strategy). Fractions containing the exosomes can be kept.

In certain example embodiments, the method yields an exosmal concentrate that is at least 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or at least 20 percent of the starting volume of biologic fluid, such as milk. In certain example embodiments, the method yields an exosmal concentrate that is about 7 percent, 7.5 percent, 8 percent, 8.5 percent, 9 percent, 9.5 percent, 10 percent, 10.5 percent, 11 percent, 11.5 percent, 12 percent, 12.5 percent, 13 percent, 13.5 percent, 14 percent, 14.5 percent, 15 percent, 15.5 percent, 16 percent, 16.5 percent, 17 percent, 17.5 percent, 18 percent, 18.5 percent, 19 percent, 19.5 percent, or about 20 percent, of the starting volume of biologic fluid, such as milk.

### Loading Exosomes

In certain example embodiments, the method further includes loading the exosomes of the formulation resulting from the method described herein, with one or more cargos. The exosomes can be loaded by any suitable method. Exemplary methods of loading the milk exosomes, such as those prepared by a method described herein, are any of those set forth in International Patent Application Publication WO2020/028439, particularly at pages 83-87.

### Exemplary Cargos

The milk exosomes can be loaded with any suitable or desired cargo(s). In some embodiments, the cargo(s) are therapeutic compounds or molecules. Exemplary cargos include, but are not limited to, DNA, RNA, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, radiation sensitizers, chemotherapeutics, imaging agents, immunogens, anti-cancer drugs, any combinations thereof, and/or the like.

In certain example embodiments, the cargo is a peptide, including but not limited to an ACT-11 peptide. In certain example embodiments, the cargo is a peptide, including but not limited to an ACT-11-minus I peptide. Other peptide cargos include those set forth in International Patent Application Publication WO2020/028439, particularly at pages 67-82 and 85 and 106-111. In some embodiments, the cargo compound is esterified, such as described in International Patent Application Publication WO2020/028439 at page 81-86. In some embodiments, the cargo compound has multiple esterifications, such as described in International Patent Application Publication WO2020/028439 at page 81-86.

Exemplary hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), purines (e.g., ATP), enzymes (e.g., creatine) and steroid hormones (e.g. estradiol, testosterone, tetrahydro testosterone, cortisol).

Exemplary immunomodulators include, but are not limited to, prednisone, azathioprine, 6-MP, cyclosporine, tacrolimus, methotrexate, interleukins (e.g., IL-2, IL-7, and IL-12) , cytokines (e.g. interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7) , cytosine phosphate-guanosine, oligodeoxynucleotides, glucans, antibodies, and aptamers).

Exemplary antipyretics include, but are not limited to, non-steroidal anti- inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Exemplary anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), temgicoluril, fabomotizole, selank, bromantane, emoxypine, azapirones, barbiturates, hydroxyzine, pregabalin, isovaleric acid, and beta blockers.

Exemplary antipsychotics include, but are not limited to, benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Exemplary analgesics include, but are not limited to, paracetamol/acetaminophen, nonsteroidal anti-inflammantories (e.g. ibuprofen, naproxen, ketoprofen, and nimesulide), COX-2 inhibitors (e.g., rofecoxib, celecoxib, and etoricoxib), opioids (e.g. morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Exemplary antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammantories (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Exemplary anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Exemplary anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., amino salicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telavancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, moxifloxacin, ciprofloxacin, levofloxacin, Gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Exemplary chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemin, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g., cisplatin, carboplatin, and oxaliplatin), gemcitabine, DNA topoisomerase I-targeting drugs (e.g., camptothecin derivatives (e.g., topotecan and irinotecan)), epidermal growth factor receptor blockade family agents (e.g., cetuximab, gefitinib), farnesyltransferase inhibitors (e.g., L-778-123), COX-2 inhibitors (e.g., rofecoxib, celecoxib, and etoricoxib), bFGF and VEGF targeting agents (e.g., bevazucimab and thalidomide), NBTXR3, Nimoral, trans sodium crocetinate, NVX-108, and combinations thereof. See also e.g., Kvols, L. K., J Nucl Med 2005; 46:187S-190S.

Exemplary immunogens, carried as cargo or attached to the external surface of the isolated exosomes could include Keyhole Limpet Hemocyanin (KLH), Concholepas Concholepas Hemocyanin (CCH), (also Blue Carrier Immunogenic Protein), Bovine Serum Albumin (BSA), Ovalbumin (OVA), and antigens used to generate immune responses to pathogens causing disease including that causing diphtheria, tetanus, pertussis, measles, mumps, rubella, hepatitis A, hepatitis B, meningococcal disease (e.g., meningitis), human papillomavirus varicella, rabies, flu, rotoviral, HIV, malarial and coronaviral disease.

### MILK EXOSOME FORMULATIONS AND KITS

Also described herein are pharmaceutical formulations that can contain an amount, effective amount, and/or least effective amount, and/or therapeutically effective amount of one or more milk exosomes, such as cargo loaded milk exosomes, described in greater detail elsewhere herein and a pharmaceutically acceptable carrier or excipient. Described in certain example embodiments herein are formulations that include exosomes, where the formulation is produced at least in part by any one of the methods of any one of the preceding paragraphs and/or described elsewhere herein, such as in the Working Examples below. Described in certain example embodiments herein are methods that include administering a formulation as described in any one of the previous paragraphs and/or elsewhere herein, such as the Working Examples below, to a subject. In some embodiments, the formulation administered to the subject includes milk exosomes, such as any of those described elsewhere herein and/or prepared by a method described elsewhere herein. In some embodiments, the milk exosomes are cargo loaded milk exosomes.

In some embodiments the subject to which the milk exosomes or formulation thereof is administered has a disease or disorder. Exemplary diseases or disorders include, but are not limited to, a cancer, a viral infection, a bacterial infection, a parasite infection, a external and internal wounds and tissue injuries, cancer, ischemic and/or hypoxic injuries (e.g. myocardial infarction, ischemic wounds and/or stroke), multiple sclerosis, psoriasis, scleroderma, acne, eczema, or a disease of the skin and/or connective tissues, cardiac diseases or disorders, neurodegenerative diseases or disorders, neurological disorders, atherosclerosis, pathologies involving epithelial permeablization and/or neovascularization (e.g., angiogenesis or vasculogenesis), respiratory distress syndrome (RDS), reperfusion injuries, dermal vascular blemish or malformation, macular degeneration, neovascularization of choriocapillaries through Bruch's membrane, diabetic retinopathy, (imflammatory and inflammation-related diseases and disorders), and radiation dermatitis.

Wounds can be chronic wounds or wounds that appear to not completely heal. Wounds that have not healed within three months, for example, are said to be chronic. Chronic wounds include, diabetic foot ulcers, ischemic, venous ulcers, venous leg ulcers, venous stasis, arterial, pressure, vasculitic, infectious, decubitis, burn, trauma-induced, gangrenous and mixed ulcers. Chronic wounds include wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed invention in wound healing and tissue regeneration can include in humans and agricultural, sports and pet animals.

Tissue injuries can result from, for example, a cut, scrape, compression wound, stretch injury, laceration wound, crush wound, bite wound, graze, bullet wound, explosion injury, body piercing, stab wound, surgical wound, surgical intervention, medical intervention, host rejection following cell, tissue or organ grafting, pharmaceutical effect, pharmaceutical side-effect, bed sore, radiation injury, radiation illness, cosmetic skin wound, internal organ injury, disease process (e.g., asthma, cancer), infection, infectious agent, developmental process, maturational process (e.g., acne), genetic abnormality, developmental abnormality, environmental toxin, allergen, scalp injury, facial injury, jaw injury, sex organ injury, joint injury, excretory organ injury, foot injury, finger injury, toe injury, bone injury, eye injury, corneal injury, muscle injury, adipose tissue injury, lung injury, airway injury, hernia, anus injury, piles, ear injury, skin injury, abdominal injury, retinal injury, eye injury, corneal injury, arm injury, leg injury, athletic injury, back injury, birth injury, premature birth injury, toxic bite, sting, injury to barrier function, injury to endothelial barrier function, injury to epithelial barrier function, tendon injury, ligament injury, heart injury, heart valve injury, vascular system injury, cartilage injury, lymphatic system injury, craniocerebral trauma, dislocation, esophageal perforation, fistula, nail injury, foreign body, fracture, frostbite, hand injury, heat stress disorder, laceration, neck injury, self-mutilation, shock, traumatic soft tissue injury, spinal cord injury, spinal injury, sprain, strain, tendon injury, ligament injury, cartilage injury, thoracic injury, tooth injury, trauma, nervous system injury, burn, burn wound, wind burn, sun burn, chemical burn, aging, aneurism, stroke, surgical radiation injury, digestive tract injury, infarct, or ischemic injury.

Cardiac diseases and disorders can include, but are not limited to, myocardial infarction, cardio myopathies (e.g., hypertrophic cardiomyopathy), arrhythmias, congestive heart failure. The regenerative effects of the provided composition may result in beneficial changes in membrane excitability and ion transients of the heart. There are many different types of arrhythmia that can lead to abnormal function in the human heart. Arrhythmias include, but are not limited to bradycardias, tachycardias, alternans, automaticity defects, reentrant arrhythmias, fibrillation, AV nodal arrhythmias, atrial arrhythmias and triggered beats, Long QT syndrome, Short QT syndrome, Brugada syndrome, premature atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supraventricular Tachycardia, Junctional rhythm, Junctional tachycardia, Premature junctional complex, Wolff-Parkinson- White syndrome, Lown-Ganong-Levine syndrome, Premature Ventricular Contractions (PVC) sometimes called Ventricular Extra Beats, alternans and discordant alternans, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, Third degree heart block, and several accessory pathway disorders (e.g., Wolff-Parkinson-White syndrome (WPW)).

Neurodegenerative and neurological disorders include, but are not limited to dementia, Alzheimer's disease, Parkinson's disease and related PD-diseases, amyotrophic lateral sclerosis (ALS), motor neuron disease, schizophrenia, spinocerebellar ataxia, prion disease, Spinal muscular atrophy (SMA), multiple sclerosis, epilepsy and other seizure disorders, and Huntington's disease.

Inflammatory diseases and inflammatory-related diseases and disorders can be asthma, eczema, sinusitis, atherosclerosis, arthritis (including but not limited to rheumatoid arthritis), inflammatory bowel disease, cutaneous and systemic mastocytosis, psoriasis, and multiple sclerosis. As used herein, the term “inflammatory disorder” can include diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some inflammatory disorders fall within one or more categories. Exemplary inflammatory disorders include but are not limited to atherosclerosis; arthritis; inflammation-promoted cancers; asthma; autoimmune uveitis; adoptive immune response; dermatitis; multiple sclerosis; diabetic complications; osteoporosis; Alzheimer's disease; cerebral malaria; hemorrhagic fever; autoimmune disorders; and inflammatory bowel disease. In some embodiments, the inflammatory disorder is an autoimmune disorder that, in some aspects, is selected from lupus, rheumatoid arthritis, and autoimmune encephalomyelitis.

In some embodiments, the inflammatory disorder is a brain-related inflammatory disorder. The term “brain-related inflammatory” disorder is used herein to refer to a subset of inflammatory disorders that are caused, at least in part, or originate or are exacerbated, by inflammation in the brain of a subject.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the cargo can optionally be present in the pharmaceutical formulation as a pharmaceutically acceptable salt. In some embodiments, the pharmaceutical formulation can include, such as an active ingredient, one or more milk exosomes, such as cargo loaded milk exosomes, described in greater detail elsewhere herein.

In some embodiments, the cargo is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s) and/or cargos.

Where appropriate, one or more milk exosomes, such as cargo loaded milk exosomes, described in greater detail elsewhere herein can be provided to a subject in need thereof as an ingredient, such as an active ingredient or agent, in a pharmaceutical formulation. As such, also described are pharmaceutical formulations containing one or more milk exosomes, such as cargo loaded milk exosomes, described in greater detail elsewhere herein include a cargo that is in the form of a pharmaceutically acceptable salt. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed. In some embodiments the active agent is a milk exosome, or a cargo loaded milk exosome. In some embodiments, the active agent includes or is the cargo of a cargo loaded milk exosome.

In certain embodiments, the milk exosome is prepared by any method described elsewhere herein. In some embodiments, the cargo loaded milk exosome is as described and/or prepared by a method as described elsewhere herein.

In some embodiments, the milk exosomes or formulations thereof are included on a material to administer the milk exosomes or formulations thereof to a subject. Non-limiting examples of materials include those that used to treat wounds such as bandages, steri-strip, sutures, staples, or grafts (e.g., skin grafts). Other exemplary materials include medical devices or implants (or components thereof). Non-limiting examples of medical implants include: limb prostheses, breast implants, penile implants, testicular implants, artificial eyes, facial implants, artificial joints, heart valve prostheses, vascular prostheses, dental prostheses, facial prosthesis, tilted disc valve, caged ball valve, ear prosthesis, nose prosthesis, pacemakers, cochlear implants, stents, shunts, catheters, filters, meshes, fillers (e.g., fat and dermal filers), and skin substitutes (e.g., porcine heterograft/pigskin, BIOBRANE, cultured keratinocytes), and/or the like.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, milk and milk products (e.g. casein, ice cream, custards, creamers, and/or the like), salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combination thereof.

### Effective Amounts

In some embodiments, the amount of the primary active agent (e.g., milk exosome, cargo loaded milk exosome, and/or cargo) and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus or virus like particle as a primary or secondary agent, e.g, as a cargo), the effective amount of virus particles can be expressed as a titer (plaque forming units per unit of volume) or as a MOI (multiplicity of infection). In some embodiments, the effective amount can be about 1×10 1 particles per pL, nL, pL, mL, or L to 1×10 20/particles per pL, nL, pL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, pL, mL, or L. In some embodiments, the effective titer can be about 1×10¹ transforming units per pL, nL, pL, mL, or L to 1×10²⁰/transforming units per pL, nL, pL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, pL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, anti-cancer drugs, immunogens, and any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, end/or exosomes, described herein or a combination thereof can be presented as a combination kit. In some embodiments, the kit includes one or more filters, tubes, devices, etc. that is used to prepare milk exosomes according to a method described herein. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, and/or exosomes described herein or any combination thereof contained therein, safety information regarding the content of the compounds, compositions, formulations (e.g., pharmaceutical formulations), particles, and/or exosomes described herein or a combination thereof contained therein, information regarding the dosages, indications for use, and/or recommended treatment regimen(s) for the compound(s) and/or pharmaceutical formulations contained therein. In some embodiments, the instructions provide direction on how to prepare milk exosomes according to a method described elsewhere herein. In some embodiments, the instructions can provide directions for administering the compounds, compositions, formulations, particles, and cells described herein or a combination thereof to a subject in need thereof.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1— Exosome Isolation from Bovine Milk

### Results

### Optimized Ultracentrifugation-Based Isolation Protocol

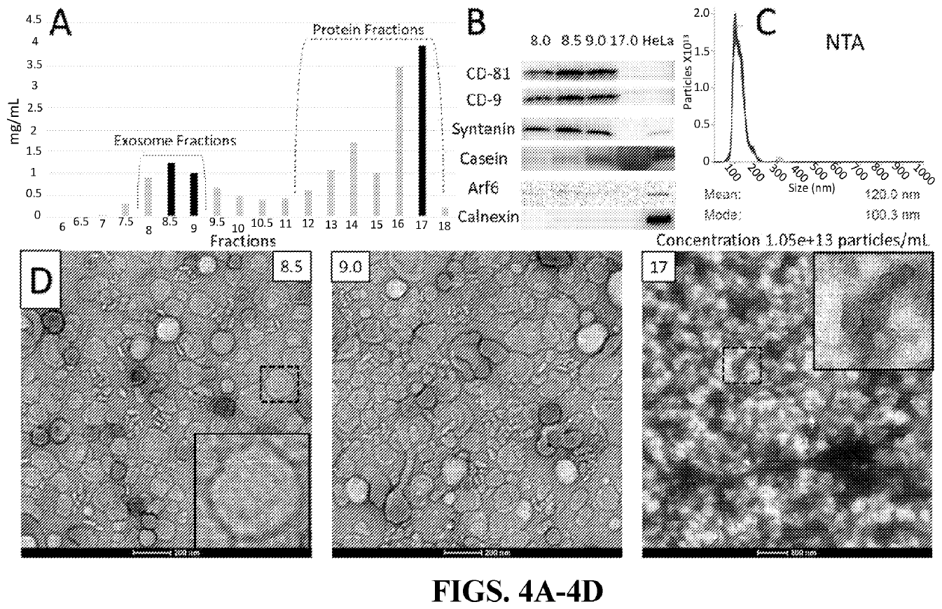

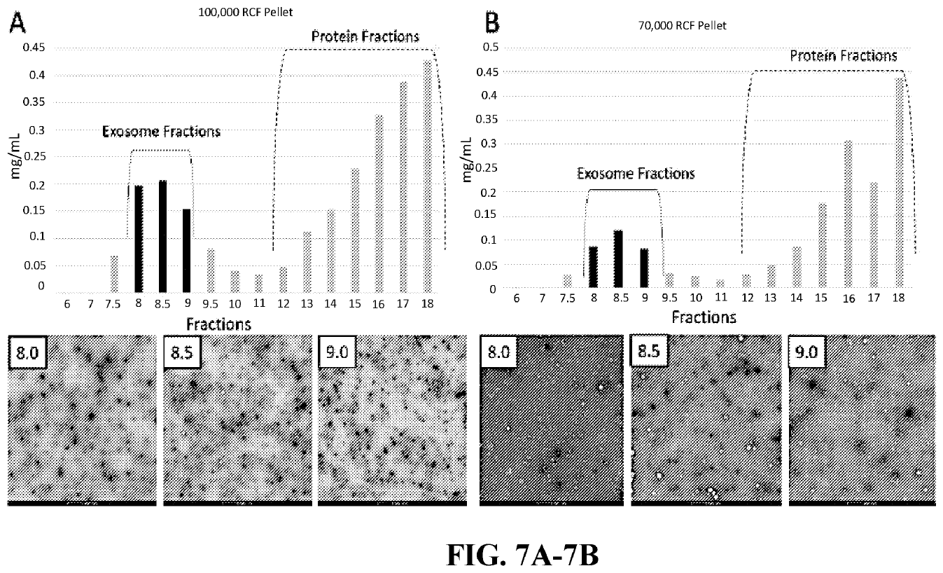

Two distinct protocols were optimized for isolation of purified exosomes from milk. The key step in each of the protocols was the timing of chemical solubilization of casein micellar structures by divalent cation chelation with 30 mM EDTA at 37° C. for 1 hour. The first protocol is referred to as the ultracentrifugation (UC)-based method (FIG. 1). The second incorporated tangential flow filtration (TFF) and is referred subsequently to as the TFF-based method (FIG. 2). In the UC-based method, high levels of exosomal yield and purity were achieved by placement of the primary chelation step prior to the final sepharose column (SEC) filtration step (FIGD. 3A-3E). The histogram in FIG. 3A shows sequential fractions collected during SEC filtration, with protein concentrations measured by nanodrop in mg/ml on the y axis. Western blotting demonstrated signals for the exosomal markers CD81, CD9, and syntenin, in tandem with the absence of bands corresponding to calnexin (a cell membrane marker), Arf6 (a microvesicle marker) and casein in the peak exosomal SEC fractions, i.e., fractions 8.5 and 9.0 (FIG. 3B). Exosomal protein markers were absent from later SEC fractions 15-18 (e.g., FIG. 3B). The Nanoparticle Tracking Analysis (NTA) graph in FIG. 3C was measured from fraction 8.5, which as shown by negative stain transmission electron microscopy (TEM) was the most enriched in exosomes (FIG. 3D). The NTA analysis indicated particle sizes consistent with exosomes (mode˜133 nm) at a concentration of about 1×10¹³ particles per mL. The TEM images in FIG. 3D show negative staining of densely packed exosomes in the peak SEC fractions. FIG. 3E shows casein macrostructures in fraction 17—a typical casein micelle found in these fractions is shown at higher magnification in the inset. In sum, TEM, NTA and Western blotting confirm the presence of pure and ultrastructurally definitive exosomes at very high density in peak SEC fractions 8.0 through 9.0, with low levels of protein signal and particulate matter corresponding to casein and casein micellar aggregates, which are seen in the later SEC fractions. Notably, from a typical starting amount of 1000 mL of milk at the beginning of the UC-based protocol, these final ultra-dense exosomal concentrates comprise an average of 75 ml (+/−10 mL)—i.e., 7.5% of the starting volume of milk (FIG. 6).

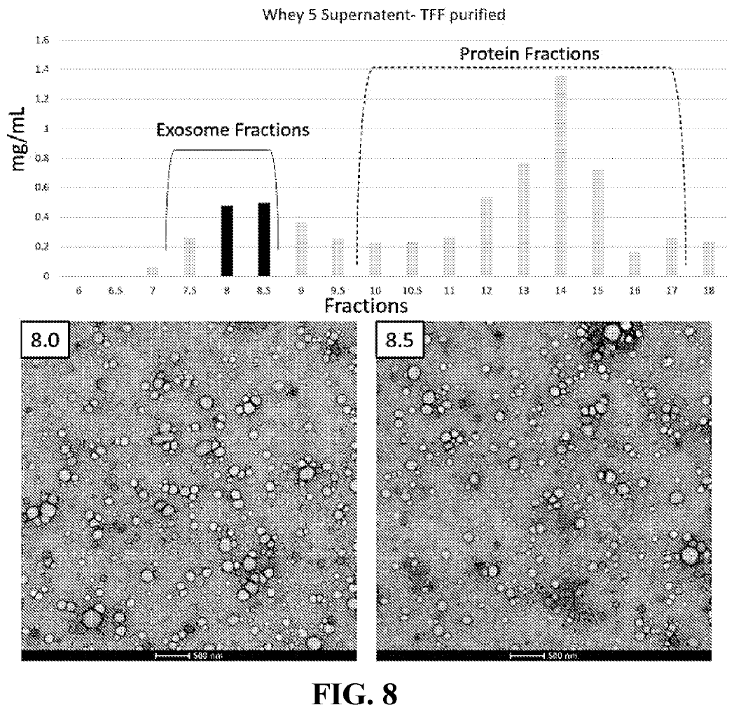

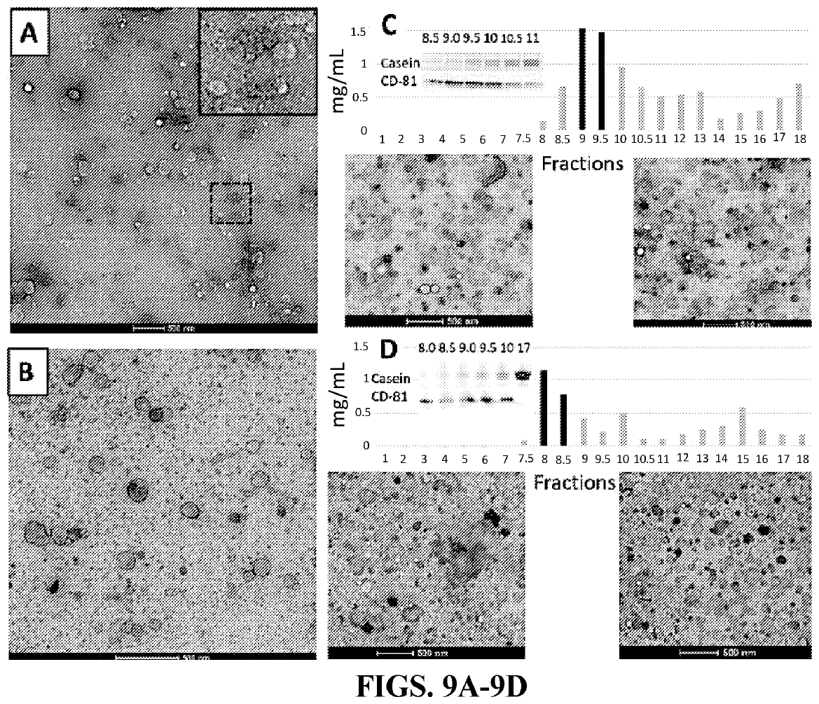

Shearing forces imparted during UC may are thought to have deleterious effects on exosome structure (Mogi K, 2018) (Lasser C, 2012). Our observations also suggest that UC may have effects on the yield and purity of exosomes from milk. The composition of pellets from the 70,000 RCF and 100,000 RCF spins, along with supernatant TFF-concentrated after the 130,000 RCF spin of the UC-based method, are shown in FIGS. 7A-7B and 8, respectively. SEC filtration of these samples followed by TEM indicate the presence of large numbers of exosomes in all of these samples. As these pellets and supernatant are discarded, the large numbers of extracellular vesicles present in these fractions are lost in the optimized UC-based method. Additionally, skipping these lower speed spins and moving directly to a 130,000 rcf spin is not an option, as the level of contaminating casein in the peak fraction resulting from SEC was high relative to the optimized method (data not shown).

### Optimized TFF-Based Isolation Protocol

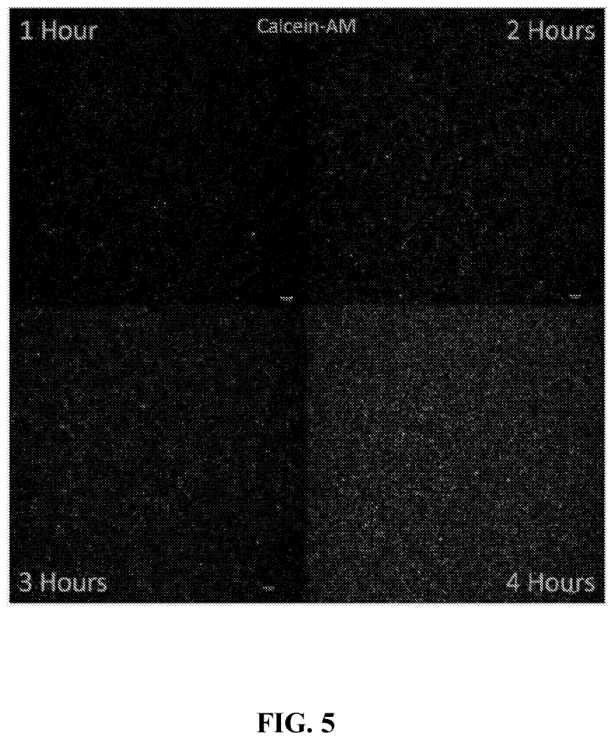

The loss of exosomes illustrated in FIGS. 7A-7B and 8 during the UC-based method led us to explore TFF as an alternate approach. The optimized protocol for TFF-based isolation of exosomes developed from our studies is summarized in FIG. 2. The histogram acquired from protein concentrations of sequential SEC fractions following TFF is shown in FIG. 4A, with protein concentrations shown on the Y axis in mg/mL. The results from the TFF-based protocol paralleled the UC-based method in many respects, although the casein solubilization step was found to be optimally placed before TFF filtration and final yields were about 100% greater than the UC-based method. Western blotting of the peak exosomal SEC fractions indicated the presence of exosomal markers CD81, CD9 and syntenin, along with the absence of microvesicle markers calnexin and Arf6, and a heavy reduction in casein in contrast to later SEC fractions e.g., fraction 17 (FIG. 4B). The NTA analysis shown is from the peak fraction 8.5 and shows a mode of 100 nm at a concentration of over 1×1013 particles per mL final solution (FIG. 4C). The TEM images below the histogram (FIG. 4D) show highly concentrated and pure exosomal solutions in the peak SEC fractions (8.5 and 9.0), while TEM of the later SEC fractions indicated high levels of casein micelle aggregates (FIG. 4E). The TFF-based protocol provided an average of 200 ml (+/−10 mL) of exosomal concentrate in its peak fractions per 1000 ml of milk, i.e., 20% of the starting volume of milk.

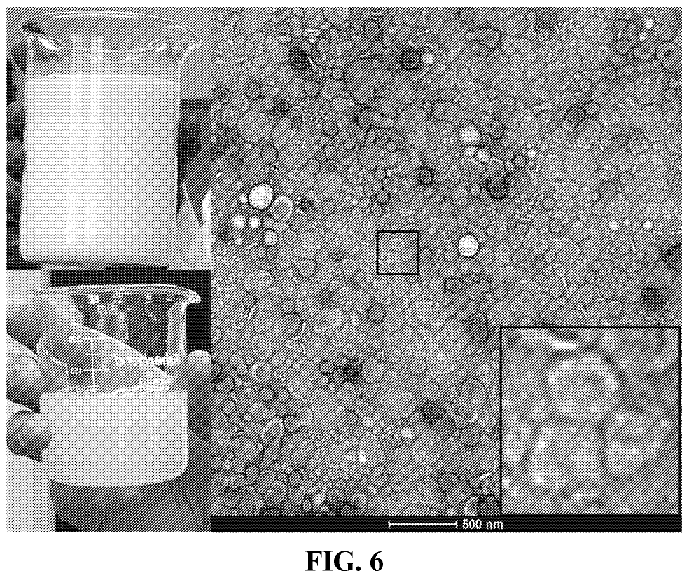

FIG. 5 shows confocal optical sections of Calcein-labeled exosomes in peak SEC fractions suspended in Hepes buffer diluted 1:10 generated by the TFF-based method. The images show uptake resulting from 1, 2, 3 and 4 hour incubations in Calcein-AM—a dye that is non-fluorescent until activation by de-esterification. The punctate fluorescent signal suggests that extracellular vesicles in our exosomal concentrates contain esterase activity and are capable of retaining de-esterified Calcein molecules. The level of Calcein signal becomes more intense with longer incubation, suggestive of the cumulative retention of dye and the structural/functional integrity of the isolated exosomes. Similar patterns of Calcein fluorescence and retention were observed in exosomes isolated using the UC-based protocol (data not shown).

Effects of Deviation from the Optimized Protocols

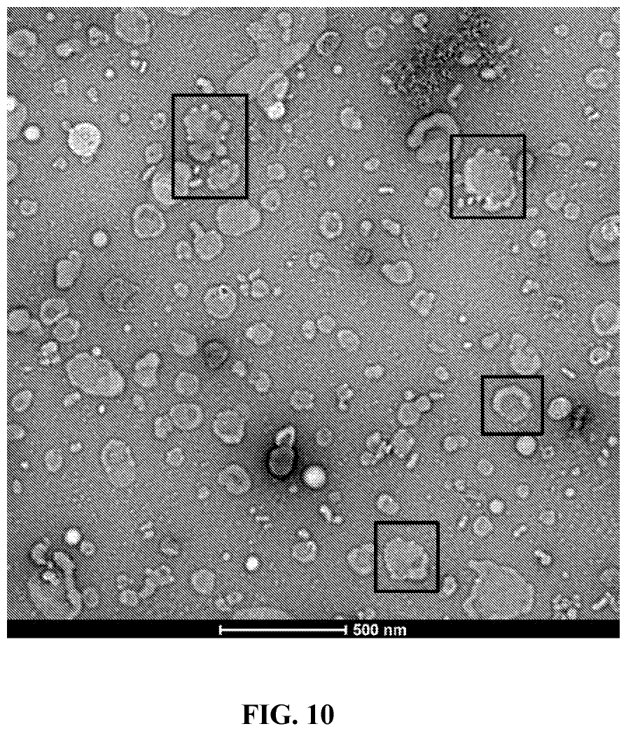

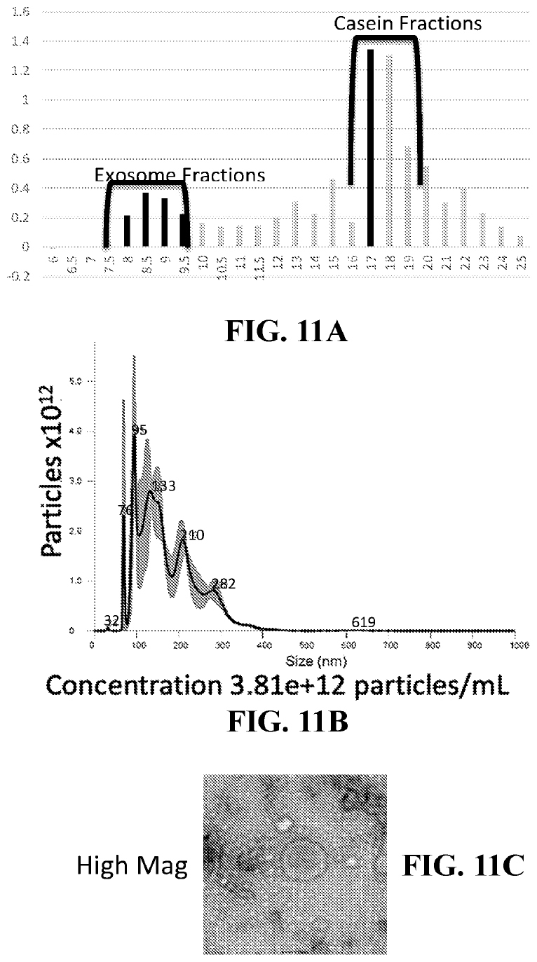

For the purpose of comparison, FIG. 9A shows the typical TEM negative stain appearance of exosomes isolated using UC-based method, but without the final casein solubilization and SEC filtration steps, as implemented in the optimized protocol. Exosomes are ultrastructurally evident, though significantly less dense than in the optimized protocol. There is also an abundance of casein micelles accompanying the exosomes. FIGS. 9B-9D illustrate examples of other sub-optimal outcomes, in terms of exosome density and casein contamination, if the SEC filtration (FIG. 9B), divalent cation chelation (FIG. 9C) and/or the 37° C. temperature (FIG. 9D) aspects of our optimized protocols are omitted. In the cases in which divalent cation chelation or 37° C. temperature incubation were not carried out, exosomal densities were decreased and casein contamination increased in peak SEC fractions, as evidenced by the ultrastructural presence of casein micelles and 20-25 kDa gel bands corresponding to casein in these same SEC fractions. We also investigated the effects of casein solubilization at EDTA concentrations lower than 30 mM, temperatures less than 37° C., incubations periods shorter than 60 minutes and implementation of the casein solubilization step at different stages of the protocol other than pre-SEC or pre-TFF for the UC-based and TFF-based methods, respectively. All these deviations from the optimal protocols resulted in peak SEC fractions exhibiting lower exosome densities and increased levels of casein contamination similar to those shown in FIGS. 9A-9D (data not shown). Incubations in 30 mM EDTA of up to 2 hours, though not deleterious, appeared to provide no further benefit in terms of exosomal yield and solubilization, whereas EDTA concentrations above 30 mM resulted in bleb-like deformations to exosome ultrastructure (FIG. 10).

### Discussion

Variation in the purity of exosomes produced by different isolation protocols, including the differential presence of EV subtypes and contamination by proteinaceous aggregates is an issue that inhibits progress in the field (Vaswani K., 2019). The problem of contamination is a particular concern when isolating exosomes from milk, where casein micelles and higher order polymeric structures containing casein, routinely co-sediment during purification of exosomal fractions (Yamauchi M, 2019). This Example at least demonstrates approaches to significantly reduce the burden of contaminant proteins in exosomal isolates. Central to the approaches is the strategic deployment of a divalent cation chelation treatment at 37° C. that promotes the solubilization of casein micellar structures. When this one hour treatment is used at specific junctures of the TFF- and UC-based protocols that are described herein, highly efficient separation of exosomes from casein-containing aggregates can be achieved. Deviation from optimized protocols, including use of concentrations of EDTA less than 30 mM and incubation at temperatures below 37° C., as well as deployment of the chelation step at stages of the methods other than those that are specified in FIGS. 1-2, result in exosomal isolates of lower purity and higher levels of contamination by milk proteins.

A further impediment to the field is the current limited ability to produce exosomes cheaply and efficiently at scale. Large starting volumes of body fluids or tissue (e.g., plasma, urine, adipose tissue), or culture media are typically required, and even then, yields of final exosomal isolates tend to be modest (Lasser C, 2012). The methods enable large volumes of purified exosomes to be produced at high density from milk in a cost-effective, straightforward series of steps. Indeed, the extent to which exosomes make up a significant fraction of milk by volume was an unexpected result from our study. The fact that milk is packed to this degree with extracellular vesicles, many of which have been reported in the literature to contain miRNAs and other molecules with informational or signaling potential (Golan-Gerstl R., 2017), places the developmentally instructive versus nutritional functions of mammalian nursing in an interesting new light.

The difference in absolute yield obtained from the TFF- and UC-based methods is notable. The ultra-dense accumulations of exosomes in peak SEC fractions resulting from the TFF-based method (e.g., FIG. 6), are equivalent to −20% of the starting volume of milk. The UC-based method yields exosomal concentrates at a still impressive −7.5% of starting volume (FIG. 3A-3E). This being said, our EM analyses suggest that the lower yield of the UC-based method may be due to the lower efficiency of this protocol as illustrated in FIGS. 7-8. In light of the larger yields obtained by the TFF-based method, it appears to be the preferred approach. This preference is reinforced by Western blotting and NTA results indicating that exosomes generated by the two methods are at comparable purity and particulate densities per unit volume. A further consideration is that protocols incorporating TFF separation may be more inherently scalable than those reliant on multiple UC steps—potentially giving a basis for the eventual industrial scale production of exosomes from milk.

The large amounts of pure exosomes generated by these methods provide an ample basis for ongoing experimentation and method testing, including the development of technical approaches to loading exosomes with cargoes such as small drugs and peptides, large macromolecular drugs, and miRNAs. Safe and efficacious drug delivery in animal models has been shown for drugs cargoed by exosomes including doxorubicin (Yang T., 2015), (Tian T., 2014), curcumin (Zhuang X., 2011) and paclitaxel (Agrawal AK, 2017), as well as siRNAs (Alvarez-Erviti L., 2011) and miRNAs (Wang F., 2018), (Momen-Heravi F., 2014). Techniques for loading exogenous molecules into exosomes reported in the literature include electroporation, sonication, freeze-thawing, extrusion and membrane saponification (Momen-Heravi F., 2014), (Haney M. J., 2015). Whilst somewhat effective, a drawback of such techniques is damage to exosomal membranes—decreasing drug retention and effective delivery of therapeutic cargoes to cells. Results from EM analyses on exosomal fractions from various iterations of our isolation protocols suggest that exosomal membranes can be sensitive to mechanical and chemical disruption (e.g., FIGS. 9A-9D and 10). Moreover, a novel and relatively gentle approach to drug loading is suggested by the Calcein retention assay in FIG. 5, wherein uptake and retention of exogenous molecules into exosomes might be enhanced by the addition of ester groups to loaded molecules. Data with short therapeutic peptides based on the connexin 43 (Cx43) carboxyl terminus, (Jiang J., 2019) suggest that esterification can be a strategy for exosomal drug loading. Embodiments, in which a cargo compound is esterified to promote exosomal uptake, are described in International Patent Application Publication WO2020/028439 at pages 81-86.

Exosomes have been utilized as drug delivery devices by numerous groups, being combined with systems such as ultrasound targeted microbubble destruction (Sun W, 2019), as simple drug carriers for neurological diseases (Yang T., 2015), as well as being engineered by designer cells to specifically target cell populations for directed delivery (Kojima R, 2018). The most promising methods of exosomes being utilized as DDDs is by engineering the exosomal surface after isolation in order to specifically upregulate desired markers for specific delivery of exosomal cargo (Si Y, 2020). Other groups have even simply applied exosomes orally with loaded cargo, representing a simple administration method that is highly effective at delivering pharmaceutical cargo (Agrawal AK, 2017). Exosomes as DDDs have been reviewed extensively in previous reviews (Vader P, 2016). The methods described and demonstrated herein can facilitate the production of industrial and clinically relevant amounts of exosomes.

### Additional Methods

### Exosome Isolation-Ultracentrifuge-Based Method

FIG. 1 summarizes the steps of the optimized UC-based protocol. Unpasteurized bovine milk at 4° C. was obtained from Homestead Creamery of Wirtz, VA. All subsequent steps up to the chelation and temperature treatment were performed at 4° C. Milk was transferred to sterile large polypropylene centrifuge tubes (Thermo Scientific, 75007585) and centrifuged at 5,000 rcf (Sorval Legend X1R centrifuge with Sorval TX-400 75003629 rotor) for 30 minutes. Fat (cream) was removed either by decanting it from the supernatant (SN) or whisking away with filter paper. The remaining SN was transferred to a new container and the pellet discarded. These steps were repeated 2-3 times to ensure defatting. Milk was then transferred to 250 mL centrifugation containers (Nalgene) and spun in at 14,500 rcf (Beckmann Coulter Avanti J-26 XP centrifuge with JLA 16.25 rotor) for 60 minutes. The SN was then transferred to 250 mL polypropylene containers (Beckmann) and centrifuged at 22,600 rcf (Beckmann Coulter Avanti J-26 XP centrifuge with JLA 16.25 rotor) for 60 minutes. After each centrifugation, the SN was decanted, the pellet discarded, and any noticeable fat was skimmed. This centrifugation, fat removal and SN decanting step was repeated at 22,600 rcf 3-4 times. The SN was then consecutively filtered through 0.45 um and 0.22 um filters (Millipore), transferred to Beckmann 355631 ultracentrifuge tubes and spun at 56,000 rcf (Beckmann Coulter Avanti J-26 XP centrifuge with JA 25.5 rotor) for 60 minutes. Following these lower speed centrifugations, the pellet was discarded and the SN transferred to a new 355631 Beckmann tube and spun at 70,000 rcf (Beckmann Coulter Optima L-100 XP Ultracentrifuge with SW.32.Ti Rotor) for 60 minutes. Subsequently, the SN was transferred to a fresh Beckmann 355631 tube, spun at 100,000 rcf (Beckmann Coulter Optima L-100 XP Ultracentrifuge with SW.32.Ti Rotor) for 60 minutes. The resulting SN was further centrifuged at 130,000 rcf (Beckmann Coulter Optima L-100 XP Ultracentrifuge with SW.32.Ti Rotor) for 120 minutes. The resulting pellet was then dissolved (pellet 10% by volume) in 2-3 ml of Hepes buffer (100 mM NaCl, 4 mM KCl, 20 mM Hepes, pH 6.7) overnight at 4° C. The following morning, the solution was triturated and aliquoted at 500 uL. These aliquots were stored at −80° C. until further use. Following thawing on ice, EDTA was added to the aliquot to a concentration of 30 mM and the solution was incubated at 37° C. for 60 minutes. The solution was then run through an IZON qEV original 70 nm sepharose column (1006881). Protein concentrations of resulting fractions were analyzed using a Nanodrop 2000 c running Nanodrop 2000 software via 260/280 spectrophotometry, using standard methods and Hepes buffer as a blank control solution. After protein quantification, samples were aliquoted and stored at −80° C. until further use.

### Exosome Isolation—Tangential Flow Filtration Based Protocol

FIG. 2 summarizes the steps of the optimized TFF-based protocol. Unpasteurized bovine milk at 4° C. was obtained from Homestead Creamery of Wirtz, VA was transferred to sterile large polypropylene centrifuge tubes (Thermo Scientific, 75007585) and processed in identical low speed centrifugation and fat skimming steps up to filtration of the resulting SN by Millipore 0.45 um and 0.22 um filters, as for the UC-based method above. The resulting solution was then treated with 30 mM EDTA at 37° C. for 60 minutes. After treatment, solution was filtered using a Repligen KrosFlo TFF system on a 500 kDa MidiKros TFF Filter (Repligen) at 10 mL/min. Once solution reached about 10% of the starting volume, the solution was further diafiltered with approximately 10× volume standard Hepes buffer- composition as for the UC-based method. In turn, once this TFF retentate reached about 20% of the starting volume, solutions were stored at −80 degrees C. prior to column separation. In other working examples, after the TFF retentate reached about 20% of the starting volume the solution was transferred to Beckmann 355631 tubes and ultracentrifuged using a SW.32.Ti rotor in a Beckmann Coulter Optima L-100XP Ultracentrifuge at 130,000 rcf for 120 minutes at 4° C. The resulting pellet was resuspended in approximately 10% starting volume (−2.5 mL) of buffer and allowed to dissolve overnight at 4° C. in this solution. The following morning, the solution was triturated and aliquoted at 500 pL volumes. After storage at -80 degrees C. or the ultracentrifugation of the retentate, solutions were then separated on an IZON qEV original 70 nm sepharose column (1006881), the resulting fractions analyzed via Nanodrop and spectrophotometry as described in the UC-based method above. After protein quantification, samples were aliquoted by fraction and stored at −80° C. until subsequent use.

### Gel Electrophoresis and Western Blotting

To prepare samples for electrophoresis, they were mixed with Lamelli's sample buffer (BioRad) containing 0.05% beta-mercaptoethanol (Thermo Fisher). Samples were then boiled for 5 minutes and 6.25 mg of protein were loaded into each lane of 4%-20% Biorad stain-free gels (BioRad-5678093). Electrophoresis was performed in standard running buffer (25 mM Tris, 192 mM Glycine, 0.1% SDS) in a Biorad module (CRITERION Cell 135BR 0030876) for 40 minutes at 200V. Stain free gel was then imaged in a Bio-Rad imager (BioRad Universal Hood III 731BR00622) using 5 minute activation imaging. Protein transfer from gels was performed in standard transfer buffer (25 mM Tris, 192 mM Glycine, 0.01% SDS) in a Bio-Rad trans-blot turbo at 25V and 1.0A for 30 minutes onto a PVDF (Millipore IPFL00010) membrane. Subsequently, the membrane was dried at room temperature (RT) for 1 hour to affix proteins. The PVDF transfer membrane was then rehydrated in methanol, washed in distilled water and blocked in 3% Fish Skin-Gelatin Extract (FSE) (Thermo Fisher) in TBST (20 mM Tris, 150 mM NaCl, 0.1% Tween-20, pH 7.6) for 1 hour at room temperature. Overnight primary antibody incubation was undertaken as directed by manufacturer instructions- diluted in 3% FSE in TBST and left overnight at 4° C. Antibodies used are: CD81 (Cell Signaling Technology, 56039S), TSG-101 (Invitrogen, MA1-23296), CD9 (Novus, NB500-494), Calnexin (EMD Millipore, AB2301), Casein (Abcam, ab166596), ARF6 (Novus, NBP1-58310), Syntenin-1 (Santa Cruz, SC-100336), with associated secondary antibodies being: Anti-Mouse (Jackson Immuno, 715-035-150) and Anti Rabbit (Southern Biotechnology, 4050-05). The membrane was then washed 5×in TBST for 5 minutes at RT on an orbital shaker (VWR Model 100 10M0219G) to remove non-bound antibody. Following washing, the membrane was incubated for 1 hour at RT in secondary antibodies diluted 1:20,000 in 1:1 TBST:3% FSE, then was washed 5× in TBST for 5 minutes on a shaker. The blot membrane was then activated by Thermo Scientific Pico activation buffer (Thermo Scientific) for 5 minutes and imaged on a Biorad imager under Chemi-detection settings. Bands were quantified using densitometry analysis in Image Lab version 6.1 (BioRad).

### Nanosight Tracker Analysis

Nanosight Tracker Analysis (NTA) analysis was performed on a Nanosight NS300. Exosomal concentrates obtained post-SEC were diluted 1:10 in Hepes buffer, then underwent bath sonication in a Branson 2510 bath sonicator for 1 minute at RT to reduce sample aggregation. Exosomes were then diluted (1:1000 to 1:10,000 depending on sample) and added to a 1 mL syringe, then set on the syringe pump and loaded into the NTA flow cell. Each sample was analyzed using a 405 nm laser with 3 consecutive 1 minute video recordings with a constant flow rate set at 10 in the NTA software (Version 3.4). Flow rates are set in the software and do not contain units. All videos were compiled and analyzed together in the NTA software and data were collected and saved in raw form.

### Confocal Microscopy Analysis

SEC exosomal concentrates were diluted 1:10 in Hepes buffer then incubated at 37° C. with Calcein-AM (Thermo Fisher Scientific C1430) at 10 uM at 1, 2, 3 or 4 hour intervals. After incubation, extravesicular dye was removed with a sepharose G50 column (USA Scientific 1415-1601) and preequilibrated with Hepes buffer. 6 uL of this solution was then dispensed onto a microscope slide (Premiere 75×25×1 mm, 9105) and coverslipped (Fisherbrand, 12541A). Calcein intensity and dye retention is particles suspended in this solution was monitored directly by optical sectioning using 63× objective (oil, 1.4 NA) on a Leica SP8 confocal microscope with 488 laser, HyD, 1AU, and scan frequency of 700 Hz for 6 fields per slide.

### Transmission Electron Microscopy-Negative Staining

Formvar-coated 200 mesh copper grids (Electron microscopy sciences, FCF200-CU) were glow discharged on a Pelco glow discharge unit (Pelco) at 0.29 mBAR for 1 minute. 0.1% poly-L-Lysine was applied to the grid for 1 minute, then excess solution wicked away with Whatmann #1 filter paper. Grids were washed 2× with 10 uL milli-Q water and excess liquid was removed with filter paper. Grids were then dried overnight at RT. Samples were loaded by applying 10 uL of prepared SEC exosomal concentrate to the grid for 5 minutes. Excess solution was wicked off with filter paper, the sample negative-stained with 10 uL uranyless stain (Electron microscopy sciences, 22409) for 1 minute at RT. Excess stain was then wicked off. The grid was then left to dry overnight at RT before transmission electron microscope (TEM) imaging. Imaging of negatively stained preparations was performed on a FEI Tecnai G20 Biotwin TEM at 120 kV and images captured using an Eagle 4K HS camera. References for Example 1

Agrawal AK, A. F. (2017). Milk derived exosomes for oral delivery of paclitaxel. Nanomedicine, 1627-1636.

Alvarez-Erviti L., S. Y. (2011). Delivery of siRNA to the mouse brain by systemic injection of targeted exosomes. Nat Biotechnol, 341-5.

An M., W. J. (2018). Comparison of an optimized ultracentrifugation method versus size-exclusion chromatography for isolation of exosomes from human serum. J Proteome Res, 3599-3605.

Antes T J, M. R. (2018). Targeting extracellular vesicles to injured tissue using membrane cloaking and surface display. J Nanobiotechnology.

Anurag, P. (2019). Exosomes from Cell Culture Conditioned Medium: Isolation by Ultracentrifugation and Characterization . Methods Mol Biol, 233-244.

Baranyai T, H. K. (2015). Isolation of exosomes from Blood Plasma: qualitative and quantitative comparison of Ultracentrifugation and Size Exclusion Chromatography Methods. PLoS One.

Bhat, M. T. (2016). Casein Proteins: structural and functional aspects. Intech.

Blaser MC, A. E. (2018). Roles and Regulation of Extracellular Vesicles in Cardiovascular Mineral Metabolism. Front Cardiovasc Med.

Boulanger, C. L. (2017). Extracellular vesicles in coronary artery disease. Nature reviews cardiology, 259.

Carrasco E, S.-H. G. (2019). The role of extracellular vesicles in Cutaneous Remodeling and Hair Follicle Dynamics. Int J Mol Sci, 2758.

Escudier. (2005). Vaccination of metastatic melanoma patients with autologous dendritic cell derived exosomes: results of the first phase I clinical trial. J transl. med, 1-13.

Golan-Gerstl R., S. Y. (2017). Characterization and biological function of milk-derived miRNAs. Mol Nutr Food Res.

Gyorgy, B. S. (2011). Membrane vesicles, current state-of-the-art: emerging role of extracellular vesicles. Cellular and molecular life sciences, 2667.

Haney M. J., K. N. (2015). Exosomes as drug delivery vehicles for Parkinson's disease therapy. J Control Release, 18-30.

Jiang J., H. D. (2019). Interaction of a Carboxyl Terminus 1 Peptide With the Connexin 43 Carboxyl Terminus Preserves Left Ventricular Function After Ischemia-Reperfusion Injury. J Am Heart Assoc.

Koh Y Q, A. F. (2018). Exosome enrichment by ultracentrifugation and size exclusion chromatography. Front Biosci (Landmark Ed), 865-874.

Kojima R, B. D.-B. (2018). Designer exosomes produced by implanted cells intracerebrally deliver therapeutic cargo for Parkinson's disease treatment. Nat Commun.

Lasser C, E. M. (2012). Isolation and characterization of RNA-containing exosomes. . J Vis Exp.

Manca S, U. B. (2018). Milk exosomes are bioavailable and distinct microRNA cargos have unique tissue distribution patterns. Sci Rep.

Mogi K, H. K. (2018). Damage-less handling of exosomes using an Ion-depletion zone in a microchannel. Anal Sci , 875-880.

Momen-Heravi F., B. S. (2014). Exosome-mediated delivery of functionally active miRNA-155 inhibitor to macrophages. Nanomedicine, 1517-27.

Mulcahy, L. P. (2014). Routes and mechanisms of extracellular vesicle uptake. Journal of Exracellular Vesicles, 241-246.

Pin Li, M. K. (2017). Progress in exosome isolation techniques. Theranostics, 789-804.

Rana, S. Z. (2011). Exosome target cell selection and the importance of exosomal tetraspanins: a hypothesis. Biochem Soc Trans, 559-562.

Rood. (2010). Comparison of three methods for isolation of urinary microvesicles to identify biomarkers of nephrotic syndrome. Kidney Int, 810-816.

Sedykh S. E., B. E. (2020). Milk Exosomes: Isolation, Biochemistry, Morphology, and Perspectives of Use. In C. J. De Bona A. G., Extracellular Vesicles and their importance in human health. Intech Open.

Si Y, K. S.-S. (2020). Targeted exosomes for drug delivery: biomanufacturing, surface tagging, and validation. Biotechnol J.

Skotland, T. S. (2017). Lipids in exosomes: current knowledge and the way forward. Progress in lipid research, 30-42.

Soares, A. M.-M. (2015). Gap junctional protein Cx43 is involved in the communication between extracellular vesicles and mammalian cells. Scientific reports, 13243.

Sun W, L. Z. (2019). Efficient exosome delivery in refractory tissues assisted by ultrasound-targeted microbubble destruction. Drug Deliv., 45-50.

Svensson, K. S. (2013). Exosome uptake depends on ERK1/2-heat shock protein 27 signaling and lipid raft mediated endocytosis negatively regulated by caveolin-1. Journal of Biological Chemistry, 17713.

Taylor DD, S. S. (2015). Methods of isolating extracellular vesicles impact down-stream analyses of their cargoes. Methods, 3-10.

Tian T., L. S. (2014). A doxorubicin delivery platform using engineered natural membrane vesicle exosomes for targeted tumor therapy. Biomaterials, 2383-90.

Tian, T. Z. (2014). Exosome uptake through clathrin-mediated endocytosis and macropinocytosis and mediating miR-21 delivery. Journal of biological chemistry, 22258.

Urbanelli, L. M. (2013). Signaling pathways in exosomes biogenesis, secretion and fat. Genes, 152-172.

Vader P, M. E. (2016). Extracellular Vesicles for drug delivery. Adv. Drug Deliv Rev, 148-156.

Vashisht M, R. P. (2017). Curcumin encapsulated in milk exosomes resists human digestion and possesses enhanced intestinal permeability in vitro. Applied Biochem Biotechnol, 993-1007.

Vaswani K, K. Y. (2017). A method for the isolation and enrichment of purified bovine milk exosomes. Reprod Biol, 341-348.

Vaswani K., M. M. (2019). A Method for the Isolation of Exosomes from Human and Bovine Milk. J Nutr Metab.

Vlassov, A. M. (2012). Exosomes: Current knowledge of their composition, biological functions, and diagnostic and therapeutic potentials. . BBA-General Subjects, 940-947.

Wang F., L. L. (2018). Exosome miR-335 as a novel therapeutic strategy in hepatocellular carcinoma. Hepatology, 940-54.

Wolf T, B. S. (2015). The intestinal transport of bovine milk exosomes is mediated by endocytosis in human colon carcinoma Caco-2 cells and rat small intestinal IEC-6 cells. J Nutr, 2201-2206.

Yamauchi M, S. K. (2019). Efficient method for isolation of exosomes from raw bovine milk. Drug Dev Ind Pharm, 359-364.

Yang T, M. P. (2015). Exosome delivered anticancer drugs across the blood-brain barrier for brain cancer therapy in Danio rerio. Pharm Res, 2003-2014.

Yang T., M. P. (2015). Exosome delivered anticancer drugs across the blood brain barrier for brain cancer therapy in Danio Rerio. Pharm Res, 2003-2014.

Zempleni J, A.-L. A. (2017). Biological Activities of extracellular Vesicles and their cargos from bovine and human milk in humans and implications for infants. J Nutr, 3-10.

Zhuang X., X. X. (2011) Treatment of brain inflammatory diseases by delivering exosome encapsulated anti-inflammatory drugs from the nasal region of the brain. Mol. Ter., 1769-1779.

### Example 2—Exosome Isolation from Human Breast Milk

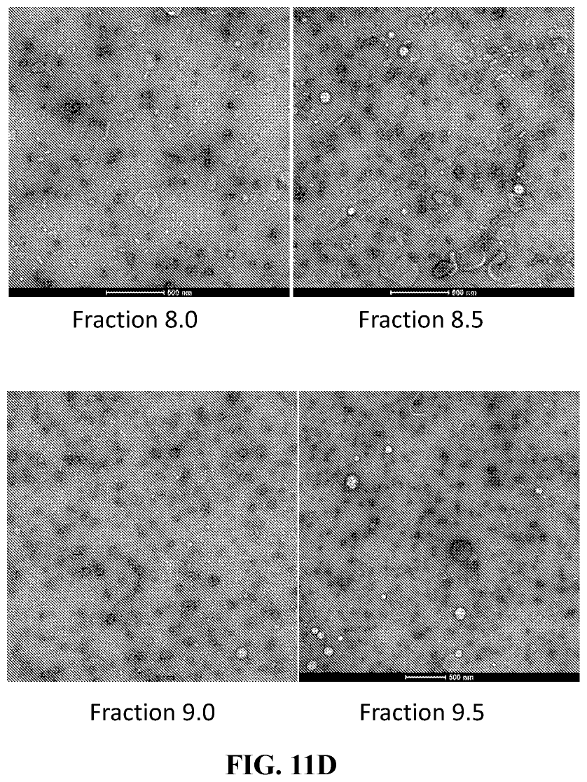

This Example at least demonstrates isolation of milk exosomes from human breast milk using the ultracentrifugation methodology described elsewhere herein, see e.g., FIG. 1. After milk collection, milk samples were stored at −80 degrees C. for 6 months prior to exosome isolation. Human milk exosomes were isolated using a UC based isolation method. See e.g., FIG. 1 and related discussion elsewhere herein. FIGS. 11A-11D show the results from isolation of exosomes from human breast milk via an embodiment of an ultracentrifugation isolation method. FIG. 11A shows a graph of the concentration of exosomes or protein in each fraction (x-axis) in mg/mL (y-axis). FIG. 11B shows Nanosight Tracker analysis data for exosome isolates. FIGS. 11C-11D show a high magnification TEM images of isolated exosomes (FIG. 11C) and representative TEM images of various exosome fractions (FIG. 11D).

### Example 3—Effect of Pre-SEC and Post-SEC storage on Isolated Milk Exosomes

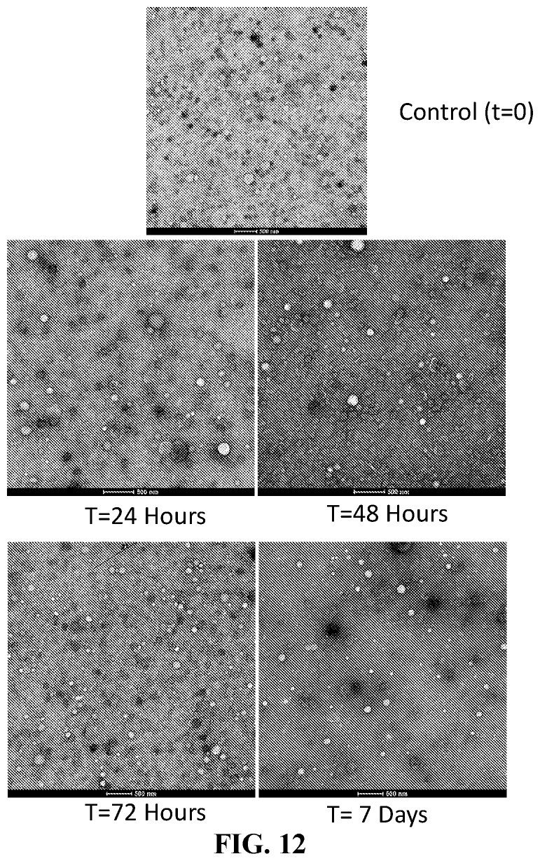

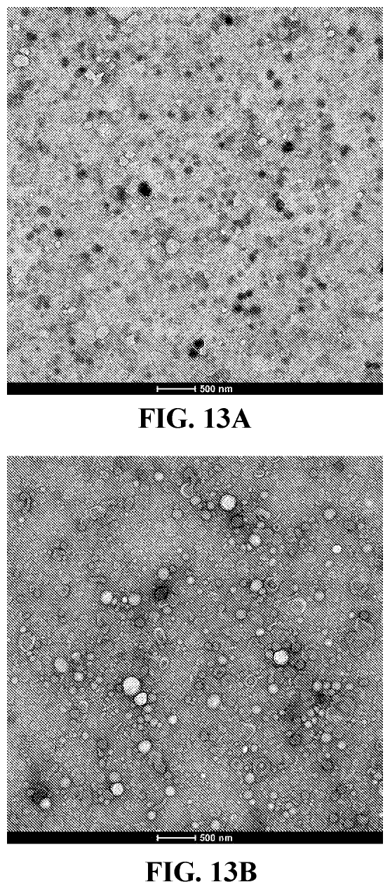

This Example evaluates the effect of pre-SEC storage and post-SEC storage on milk exosomes. Two separate analyses were used, namely storing Post-SEC and storage Pre-SEC. Without being bound by theory, Pre-SEC storage is important as it is believed the exosomes are more stable prior to gel separation. For the results shown in FIG. 12, samples were stored at −80 degrees C. post-TFF but prior to SEC for up to 3 months. SEC was then performed, and samples were stored at 4 degrees C. for the period of time indicated under each representative TEM image in FIG. 12. For the results shown in FIGS. 13A-13B, samples were isolated fresh samples that had no −80 degrees storage (FIG. 13A) at al or were stored at −80 degrees C. post-TFF but pre-SEC for 6 months prior to SEC processing (FIG. 13B). TEM of 6 month sample at least demonstrates that long term storage prior to the SEC processing is viable. It is contemplated that use of cryoprotectants including sucrose, lactose or trehalose can also be used for storage of our isolated exosomes at −80 degrees C.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A method of isolating exosomes from a biological fluid, the method comprising:
a. centrifuging a biological fluid under conditions suitable to separate fats from one or more other components of the biological fluid;
b. removing the separated fats from the biological fluid;
c. after step (b) centrifuging the remaining biological fluid one or more times and skimming any noticeable separated fats after each centrifuging in step (c);
d. filtering the remaining biological fluid after step (c)
e. optionally performing one or more ultracentrifugation steps after (d);
f. chelating divalent cations with about 10 mM to about 100 mM EDTA at about 30-42 degrees Celsius optionally after (d) or optionally (e) and optionally for about 15-120 minutes; and
g. after (f), optionally performing tangential flow filtration to obtain a retentate, wherein the retentate is optionally ultracentrifuged via one or more ultracentrifugation steps or stored at −80degrees C., and separating out fractions of the retentate, optionally via column separation, after the retentate is optionally ultracentrifuged or stored at −80 degrees C.,
wherein the method comprises step (e) or step (g) but not both.
   2. The method of aspect 1, wherein chelating divalent cations occurs with about 30 mM EDTA.
   3. The method of any one of aspects 1-2, wherein chelating divalent cations occurs at about 37 degrees Celsius.
   4. The method of any one of aspects 1-3, wherein the biological fluid is mammalian milk.
   5. The method of any one of aspects 1-4, wherein the biological fluid is unpasteurized.
   6. The method of any one of aspects 1-5, wherein steps (a) and (b) together are repeated 1-5 times.
   7. The method of any one of aspects 1-6, wherein step (a), (b), (c), (d), (e), (g), or any combination thereof is performed at about 4 degrees Celsius.
   8. The method of any one of aspects 1-7, wherein (a) comprises centrifuging the biological fluid at about 2,000-3,000 rcf.
   9. The method of any one of aspects 1-8, wherein (a) comprises centrifuging the biological fluid at about 2,500 rcf.
   10. The method of any one of aspects 1-9, wherein step (a) is repeated 1-3 times.
   11. The method of any one of aspects 1-10, wherein (b) comprises a first centrifugation followed by a second centrifugation.
   12. The method of aspect 11, wherein the first centrifugation comprises centrifuging the remaining biological fluid at about 13,500-15,500 rcf for about 45-75 minutes.
   13. The method of any one of aspects 11-12, wherein the first centrifugation comprises centrifuging the remaining biological fluid at about 14,500 rcf for about 60 minutes.
   14. The method of any one of aspects 11-13, wherein the second centrifugation is performed on the biological fluid remaining after the first centrifugation and wherein the second centrifugation is performed at about 24,800-26,800 rcf for about 45-75 minutes.
   15. The method of aspect 14, wherein the second centrifugation is performed on the biological fluid remaining after the first centrifugation and wherein the second centrifugation is performed at about 25,800 rcf for about 60 minutes.
   16. The method of any one of aspects 11-15, wherein the second centrifugation is repeated 1-3 times with each repetition being performed on the remaining biological fluid from the centrifugation immediately prior.
   17. The method of any one of aspects 1-16, wherein (d) comprises filtering the remaining biological fluid through one or more filters in series ranging from about a 0.45 micron filter to about a 0.22 micron filters.
   18. The method of aspect 17, wherein (d) comprises filtering the remaining biological fluid through an about 0.45 micron filter followed by filtering the remaining biological fluid through an about 0.22 micron filter.
   19. The method of any one of aspects 1-18, wherein (e) comprises 2 or more serial ultracentrifugation steps, wherein each step is performed on the remaining biological fluid from the prior ultracentrifugation.
   20. The method of aspect 19, wherein (e) comprises an ultracentrifugation step performed at about 45,000-55,000 rcf, an ultracentrifugation step performed at about 65,000-75,000 rcf, an ultracentrifugation step performed at about 90,000-110,000 rcf, or a combination thereof
   21. The method of any one of aspects 19-20, wherein (e) comprises an ultracentrifugation step performed at about 50,000 rcf, an ultracentrifugation step performed at about 70,000 rcf, an ultracentrifugation step performed at about 100,000 rcf, or a combination thereof
   22. The method of any one of aspects 19-21, wherein the one or more of the one or more ultracentrifugation steps are each performed for about 45-75 minutes.
   23. The method of any one of aspects 19-22, wherein the one or more of the one or more ultracentrifugation steps are each performed for about 60 minutes.
   24. The method of any one of aspects 19-23, wherein (e) comprises a final ultracentrifugation step performed at about 115,000-145,000 rcf, for about 90-150 minutes and wherein the resulting fluid is discarded, and the remaining pellet is resuspended in a suitable volume of a suitable solution prior to (f).
   25. The method any one of aspects 1-24, wherein (e) comprises a final ultracentrifugation step performed at about 130,000 rcf, for about 120 minutes and wherein the resulting fluid is discarded, and the remaining pellet is resuspended in a suitable volume of a suitable solution prior to (f).
   26. The method of any one of aspects 1-25, wherein the tangential flow filtration of (g) is performed using ultrafiltration membrane with a cutoff ranging from about 250 kDa to about 750 kDa. 27. The method of any one of aspects 1-26, wherein the tangential flow filtration of (g) is performed using a 250 kDa ultrafiltration membrane.

28. The method of any one of aspects 1-27, wherein the tangential flow filtration of (g) is performed at a flow rate of about 5-15 mL per minute.

29. The method of any one of aspects 1-28, wherein the tangential flow filtration of (g) is performed at a flow rate of about 10 mL per minute.
30. The method of any one of aspects 1-29, wherein in step (g), when the amount of remaining biological fluid reaches about ten percent of its starting volume before tangential flow filtration the retentate is diafiltered with a suitable buffer.
31. The method of aspect 30, further comprising ultracentrifuging the retentate when the retentate reaches about 20 percent of the starting diafiltration amount.
32. The method of aspect 31, wherein the ultracentrifugation is performed at about 115,000-145,000 rcf for about 90-150 minutes at about 4 degrees Celsius.
33. The method of any one of aspects 31-32, wherein the ultracentrifugation is performed at about 130,000 rcf for about 120 minutes at about 4 degrees Celsius.
34. The method of aspect 30, wherein the retentate is stored at −80 degrees C. after the retentate reaches about 20 percent of the starting diafiltration amount and prior to column separation.
35. The method of any one of aspects 1-34, wherein the method yields an exosmal concentrate that is at least 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or at least 20 percent of the starting volume of milk.
36. The method of any one of aspects 1-35, further comprising loading the exosomes of the formulation resulting from the method of any one of the preceding claims, with one or more cargos.
37. A formulation comprising exosomes, wherein the formulation is produced at least in part by a method of any one of aspects 1-36.
38. The formulation of aspect 37, wherein one or more of the exosomes are loaded with one or more cargos.
39. A method comprising:
administering a formulation as in aspect 38 to a subject in need thereof.
   40. The method of aspect 39, wherein the one or more cargos are therapeutic cargos.

What is claimed is:

1. A method of isolating exosomes from a biological fluid, the method comprising:
a. centrifuging a biological fluid under conditions suitable to separate fats from one or more other components of the biological fluid;
b. removing the separated fats from the biological fluid;
c. after step (b) centrifuging the remaining biological fluid one or more times and skimming any noticeable separated fats after each centrifuging in step (c);
d. filtering the remaining biological fluid after step (c)
e. optionally performing one or more ultracentrifugation steps after (d);
f. chelating divalent cations with about 10 mM to about 100 mM EDTA at about 30-42 degrees Celsius after (d) or optionally (e) and optionally for about 15-120 minutes; and
g. after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C., wherein the method comprises step (e) or step (g) but not both.

2. The method of claim 1, wherein chelating divalent cations occurs with about 30 mM EDTA.

3. The method of claim 1, wherein chelating divalent cations occurs at about 37 degrees Celsius.

4. The method of claim 1, wherein the biological fluid is mammalian milk.

5. The method of claim 4, wherein the biological fluid is unpasteurized.

6. The method of claim 1, wherein steps (a) and (b) together are repeated 1-5 times.

7. The method of claim 1, wherein step (a), (b), (c), (d), (e), (g), or any combination thereof is performed at about 4 degrees Celsius.

8. The method of claim 1, wherein (a) comprises centrifuging the biological fluid at about 2,000-3,000 rcf.

9. The method of claim 1, wherein (a) comprises centrifuging the biological fluid at about 2,500 rcf.

10. The method of claim 1, wherein step (a) is repeated 1-3 times.

11. The method of claim 1, wherein (b) comprises a first centrifugation followed by a second centrifugation.

12. The method of claim 11, wherein the first centrifugation comprises centrifuging the remaining biological fluid at about 13,500-15,500 rcf for about 45-75 minutes.

13. The method of claims 11, wherein the first centrifugation comprises centrifuging the remaining biological fluid at about 14,500 rcf for about 60 minutes.

14. The method of claim 11, wherein the second centrifugation is performed on the biological fluid remaining after the first centrifugation and wherein the second centrifugation is performed at about 24,800-26,800 rcf for about 45-75 minutes.

15. The method of claim 14, wherein the second centrifugation is performed on the biological fluid remaining after the first centrifugation and wherein the second centrifugation is performed at about 25,800 rcf for about 60 minutes.

16. The method of claim 11, wherein the second centrifugation is repeated 1-3 times with each repetition being performed on the remaining biological fluid from the centrifugation immediately prior.

17. The method of claim 1, wherein (d) comprises filtering the remaining biological fluid through one or more filters in series ranging from about a 0.45 micron filter to about a 0.22 micron filters.

18. The method of claim 17, wherein (d) comprises filtering the remaining biological fluid through an about 0.45 micron filter followed by filtering the remaining biological fluid through an about 0.22 micron filter.

19. The method of claim 1, wherein (e) comprises 2 or more serial ultracentrifugation steps, wherein each step is performed on the remaining biological fluid from the prior ultracentrifugation.

20. The method of claim 19, wherein (e) comprises an ultracentrifugation step performed at about 45,000-55,000 rcf, an ultracentrifugation step performed at about 65,000-75,000 rcf, an ultracentrifugation step performed at about 90,000-110,000 rcf, or a combination thereof.

21. The method of claim 19, wherein (e) comprises an ultracentrifugation step performed at about 50,000 rcf, an ultracentrifugation step performed at about 70,000 rcf, an ultracentrifugation step performed at about 100,000 rcf, or a combination thereof

22. The method of claim 19, wherein the one or more of the one or more ultracentrifugation steps are each performed for about 45-75 minutes.

23. The method of claim 19, wherein the one or more of the one or more ultracentrifugation steps are each performed for about 60 minutes.

24. The method of claim 1, wherein (e) comprises a final ultracentrifugation step performed at about 115,000-145,000 rcf, for about 90-150 minutes and wherein the resulting fluid is discarded, and the remaining pellet is resuspended in a suitable volume of a suitable solution prior to (f).

25. The method of claim 1, wherein (e) comprises a final ultracentrifugation step performed at about 130,000 rcf, for about 120 minutes and wherein the resulting fluid is discarded, and the remaining pellet is resuspended in a suitable volume of a suitable solution prior to (f).

26. The method of claim 1, wherein the tangential flow filtration of (g) is performed using ultrafiltration membrane with a cutoff ranging from about 250 kDa to about 750 kDa.

27. The method of claim 1, wherein the tangential flow filtration of (g) is performed using a 250 kDa ultrafiltration membrane.

28. The method of claim 1, wherein the tangential flow filtration of (g) is performed at a flow rate of about 5-15 mL per minute.

29. The method of claim 1, wherein the tangential flow filtration of (g) is performed at a flow rate of about 10 mL per minute.

30. The method of claim 1, wherein in step (g), when the amount of remaining biological fluid reaches about ten percent of its starting volume before tangential flow filtration the retentate is diafiltered with a suitable buffer.

31. The method of claim 30, further comprising ultracentrifuging the retentate when the retentate reaches about 20 percent of the starting diafiltration amount.

32. The method of claim 31, wherein the ultracentrifugation is performed at about 115,000-145,000 rcf for about 90-150 minutes at about 4 degrees Celsius.

33. The method of claim 31, wherein the ultracentrifugation is performed at about 130,000 rcf for about 120 minutes at about 4 degrees Celsius.

34. The method of claim 30, wherein the retentate is stored at −80 degrees C. after the retentate reaches about 20 percent of the starting diafiltration amount and prior to column separation.

35. The method of claim 1, wherein the method yields an exosmal concentrate that is at least 7.5 percent, 8 percent, 8.5 percent, 9 percent, 9.5 percent, 10 percent, 10.5 percent, 11 percent, 11.5 percent, 12 percent, 12.5 percent, 13 percent, 13.5 percent, 14 percent, 14.5 percent, 15 percent, 15.5 percent, 16 percent, 16.5 percent, 17 percent, 17.5 percent, 18 percent, 18.5 percent, 19 percent, 19.5 percent, or at least 20 percent of the starting volume of milk.

36. The method of claim 1, further comprising loading the exosomes of the formulation resulting from the method of any one of the preceding claims, with one or more cargos.

37. A formulation comprising exosomes, wherein the formulation is produced at least in part by a method of any one of claim 1-36.

38. The formulation of claim 37, wherein one or more of the exosomes are loaded with one or more cargos.

39. A method comprising:
administering a formulation as in claim 38 to a subject in need thereof.

40. The method of claim 39, wherein the one or more cargos are therapeutic cargos.
